(19) United States
(12) Patent Application Publication
(10) Pub. No.: US 2024/0237637 A1
(43) Pub. Date: Jul. 18, 2024

(54) POACEAE POLLEN PRESERVATION AND STABILIZATION METHOD

(71) Applicant: Accelerated Ag Technologies, LLC, Ames, IA (US)

(72) Inventors: George W. Singletary, Ankeny, IA (US); Jason Cope, Ankeny, IA (US); Mark Gee, West Lafayette, IN (US); Mark E. Westgate, Ames, IA (US); Federico Valverde, Polk City, IA (US); Diego Viteri, San Sebastian, PR (US); Marc Albertsen, Johnston, IA (US)

(73) Assignee: Accelerated Ag Technologies, LLC, Ames, IA (US)

(21) Appl. No.: 18/413,685

(22) Filed: Jan. 16, 2024

Related U.S. Application Data
(60) Provisional application No. 63/439,378, filed on Jan. 17, 2023.

(51) Int. Cl. A01N 3/00; A01H 1/02

(52) U.S. Cl. CPC A01N 3/00; A01H 1/02

(57) ABSTRACT

Described are methods of Poaceae pollen preservation which improve the viability and longevity of such pollen for use in pollinating plants. In addition, methods of thawing and stabilization of cryogenically stored pollen are described. Methods of statically drying pollen in preparation for cryogenic, refrigerated, or ambient storage that help preserve high pollen fertility levels are described. In addition, the requirement for a specific relative humidity range during drying is demonstrated. Using a rate of drying that is optimized to predict the pollen moisture content is used to maximize pollen fertility post-storage. In addition, the consideration of evaporative demand at the time of preparing pollen for storage is demonstrated to be important to maximizing post-storage fertility.

### CROSS REFERENCE TO RELATED APPLICATIONS

This application claims priority from U.S. Provisional Patent Application No. 63/439,378 filed Jan. 17, 2023 and titled Long Term Pollen Preservation and Re-Acclimation Method. The entire contents of U.S. Provisional Patent Application No. 63/439,378 are hereby incorporated by reference.

### FIELD OF THE INVENTION

This invention relates generally to a novel method of Poaceae pollen preservation and storage. In addition, the invention relates to a method of stabilizing previously cryogenically stored Poaceae pollen to extend its viability for use in successfully pollinating Poaceae plants.

### BACKGROUND

The current invention has application to the field of Poaceae pollen longevity and viability. Pollen longevity varies significantly among Poaceae species and is significantly influenced by environmental conditions, most notably temperature and relative humidity (RH). Pollen, which is naturally shed from the flowers or flowering structures of angiosperms, is subject to rapid loss of viability once it is shed from the plant. Viability can be lost in minutes to hours depending on species and environmental conditions. Exposure to dry air and high temperature is particularly detrimental to Poaceae pollen viability and longevity once it is shed from the plant. Thus, under natural field conditions, Poaceae pollen has a limited lifespan during which it remains viable. Pollen from the Poaceae (Gramineae) family of plants, commonly referred to as grasses, is particularly vulnerable and short-lived (Barnabas & Kovacs (1997) In: Pollen Biotechnology For Crop Production And Improvement. (1997). Sawhney, V. K., and K. R. Shivanna (eds). Cambridge University Press. pp. 293-314). This family of plants includes many economically important cereal crops, including maize. Methods to store Poaceae pollen while retaining and extending its viability are of significant value to the agricultural industry.

Specifically, if pollen collected from Poaceae plants can be stored in a viable state for a period of time, this pollen may be used to pollinate one or more female flowers as desired in a number of advantageous ways. In particular, intentional pollinations can be conducted using technologies outlined in U.S. Pat. Nos. 10,905,060; 11,849,686; and 10,398,099. Using stored pollen allows for pollination which is not dependent on active pollen shed, temporal synchrony with pistil (female flower) receptivity, use of male sterility, and/or physical isolation from other pollen sources. Currently, many Poaceae species rely on self-pollination or cross pollination by neighboring plants to produce fertile seed or grain. Typically in the agricultural hybrid seed industry, mechanical, physical, and/or genetic interventions are required to ensure female plants are cross pollinated, and not self-pollinated, so that pollen of a specific genetic constitution is employed to produce hybrid seed. Such measures, for example, are used routinely to produce hybrid maize, wheat, and rice seed.

Many attempts have been made to preserve Poaceae pollen and extend its viability beyond the time the pollen would remain viable if left exposed to uncontrolled ambient conditions. Among the grasses, studies with maize are exemplary of the progress made in pollen preservation. Many types of treatments have been tested for maintaining or extending maize pollen viability and/or fertility. Among them, the favorability of treating and/or storing maize pollen at high humidity and/or cold temperature has been reported by many.

Among the earliest accounts of maize pollen preservation (Andronescu, Demetrius I., The physiology of the pollen of Zea mays with special regard to vitality. Thesis for degree of Ph.D. University of Illinois. 1915), it was reported that in the absence of controlled environmental storage conditions, maize pollen died in two to four hours. By raising the RH of the storage environment, the pollen's viability was maintained for 48 hours. Moreover, storage at low temperature (e.g., 8-14° C.) had a stimulative effect upon the viability of the pollen.

Even when RH is not controlled during storage, maize pollen held at low temperature (e.g., 2-7° C. for 3-120 hours) can more than double its in vitro germinability compared to initial, pre-storage vitality or compared to storage at 35° C. (Pfahler, P. L. and Linskens, H. F., (1973) Planta, 111(3), pp. 253-259; Frova, C. B. and Feder, W. A., (1979) Ann Bot, 43(1), pp. 75-79). When high RH (90%) and low temperature (4° C.) during storage are combined for pollen treatment, germination of maize pollen on artificial media remains good, to fair, for eight days (Sartoris, G. B., (1942) Am J Bot, pp. 395-400). Storage of maize pollen under the same conditions for eight days also allows the pollen to remain fertile, albeit at a reduced level, and capable of forming kernels on ears following pollination (Jones, M. D. and Newell, L. C., (1948) J Amer Soc Agron 40:195-204).

Field conditioning maize pollen at high RH and low temperature commonly help revive pollen of low viability and/or extend its longevity, whereby at least limited seed formation occurs following pollination of ears (U.S. Pat. Nos. 10,575,517 and 11,344,027, and U.S. patent application Ser. No. 18/377,964). But the stimulative effect of low temperature storage on fertility is not always observed (Walden, D. B., (1967) Crop Science, 7(5), pp. 441-444) and if the pollen becomes dehydrated to excessive levels, pollen tube formation on artificial media and silks can be markedly reduced (Hoekstra, FA. (1986) In: Membranes, Metabolism and Dry Organisms. (Ed., A C Leopold), pp. 102-122, Comstock Publishing Associates, Ithaca, NY; Barnabas, B. and Fridvalszky, L., (1984) Acta Bot Hung 30:329-332).

Although high humidity and low temperature slow the temporal decay of viability during storage of Gramineae pollen, optimizing these environmental conditions for preservation only postpones the complete loss of viability and fertility. Methods in addition to regulating humidity and temperature are needed to further enhance the longevity of stored pollen so that it can be used in commercial practice of supplemental pollination.

In some cases, it may be desirable to dehydrate or hydrate Poaceae pollen to specific moisture levels. Dehydration can be achieved by vacuum drying or exposing pollen to an RH and temperature (i.e., vapor pressure deficit) that causes water to diffuse out of the pollen. In contrast, hydration can be achieved by exposing pollen to an environment with an RH and temperature that causes the pollen to absorb moisture. In both cases, dehydration or hydration of pollen may include the use of a chamber with gas flow to expose the pollen to a dehydrating or hydrating environment. Vapor pressure deficits (VPDs) favorable for pollen drying can be produced in a number of ways, such as with desiccants, mechanical equipment designed to control temperature and RH in an enclosed chamber and with saturated salt solutions held in a closed space (Jackson, M. A. and Payne, A. R. (2007) Biocontrol Sci Techn, 17(7), pp. 709-719); Greenspan, L., (1977) J Res Nat Bur Stand, 81(1), pp. 89-96).

Previous published work focused on dehydration and preservation of sugarcane pollen used storage at low temperature under vacuum with a small amount of CaCl₂ desiccant present (Sartoris, G. B. (1942) Am J Bot, pp. 395-400). The pollen remained dry throughout storage, as desired, but use of low pressure was not as favorable as storage at normal atmospheric pressure. The behavior of corn pollen is very similar to that of sugarcane. The literature has described more direct attempts at dehydration, which have incubated corn pollen in conditions of established or recorded RH and temperature. This literature shows that maize pollen can be dehydrated to very low levels (e.g., 7-10% pollen water content) and still possess an ability, albeit reduced, to effect seed formation following pollination of ears (Barnabas, B., et al. (1988) Euphytica, 39(3), pp. 221-225; U.S. Pat. No. 5,596,838).

Dehydration of pollen is commonly performed ahead of freezing for storage and preservation at very low temperatures. As practiced with maize, fresh pollen is dehydrated at room temperature in a vacuum chamber, humidity incubator, or simply with air-drying or mild heat (U.S. Pat. No. 5,596,838; Barnabas, B. and Rajki, E. (1981). Ann Bot, 48(6), pp. 861-864; Connor, K. F. and Towill, L. E. (1993) Euphytica, 68(1), pp. 77-84). Upon thawing after short or long term storage, cryopreserved pollen can be viable and fertile, but only for a very short time. In addition, fertility is not always exhibited and some members of the Poaceae family, such as maize, sorghum, rye, barley, oat, and wheat, can be difficult to cryopreserve (Collins, F. C., et al. (1973) Crop Sci, 13(4), pp. 493-494). One explanation offered for this recalcitrance is excess drying or aging of the pollen (Collins, F. C., et al. (1973) Crop Sci, 13(4), pp. 493-494). It is evident that pollen quality can be affected by prevailing environmental conditions during floral development, pollen maturation, and anthesis (Shivanna, K. R., et al. (1991) Theor Appl Genet 81(1), pp. 38-42; Schoper, J. B., et al. (1987) Crop Sci, 27(1), pp. 27-31; Herrero, M. P. and Johnson, R. R. (1980) Crop Sci, 20(6), pp. 796-800). Pollen stressed in these ways could exhibit a reduced propensity to withstand the rigors of dehydration and freezing for cryopreservation. A need exists to overcome this problem and make cryopreservation and subsequent stabilization of Poaceae pollen more attainable and routine so this form of pollen preservation can be implemented in a predictable way on a commercial scale.

Desiccation is known to have a direct impact on pollen viability. Barnabas ((1985) Ann Bot 55:201-204) and Fonseca and Westgate ((2005) Field Crops Research 94: 114-125) demonstrated that freshly harvested maize pollen could survive a reduction in original water content of approximately 50%, but few pollen grains demonstrated viability or a capacity for normal pollen tube formation with an additional water loss beyond that level. Early work by Barnabas and Rajki ((1976), Euphytica 25: 747-752) demonstrated that pollen with reduced water content would retain viability when cryogenically stored at −196° C. Subsequent work (Barnabas & Rajki (1981) Ann Bot 48:861-864) demonstrated that such partially desiccated maize pollen grains stored at −76° C. or −196° C. also could successfully fertilize receptive female flowers. Other methods of storing pollen for varying periods of time are known in the art, including freeze-drying, vacuum-drying, and storage in organic non-polar solvents. Limitations in the scalability of these pollen preservation techniques combined with the complex, non-portable equipment requirements render these techniques impractical for use with large volumes of pollen required for field-scale applications. For example, the ability to create a vacuum chamber large enough for production-level field pollination preservations would have required a much larger vacuum chamber capable of rapidly changing pressure levels. Production-level parent increase fields are typically an acre or more, while hybrid production fields are typically 10 acres or more in size. Such fields require a considerable amount of pollen and thus a large vacuum chamber would be needed. A chamber of these specifications would require the ability to pump down to a pressure of 5 Torr (0.67 kPa) or less, with the added ability to rapidly up cycle and down cycle this level of pressure. As the physical volume of the sample increases, the ability to generate and cycle at 5 Torr (0.67 kPa) efficiently begins to go beyond what mechanical pumps can generate. In addition, storage of pollen in organic solvents creates hazardous chemical requirements.

U.S. Pat. No. 5,596,838 from Greaves, et al., discloses a method of storing pollen that involves a reduction in moisture level by exposing pollen to reduced atmospheric pressures prior to storage. This technique prepared small quantities of pollen, such as from a single maize plant, for subsequent storage under sub-zero conditions. The methodology and mechanical system requirements, however, do not enable storing pollen in quantities large enough to enable commercial seed production or grain production applications. These requirements effectively negated any opportunity to advance the technology beyond research level investigations. In addition, these methods of preserving pollen do not allow for the stabilization of cryopreserved pollen after it is removed from frozen storage in such a way that the pollen remains viable for more than a very short period of time. Typically, cryogenically preserved and thawed Poaceae pollen remains viable for only a few minutes, allowing it to be used in hand pollinations immediately following its thawing, but after a brief period of viability, it rapidly dies. Accordingly, methods are required that allow for the stabilization of previously frozen pollen such that the thawed pollen can be used for several hours or even days following its removal from cryogenic conditions.

There are published protocols reported for cryopreservation of maize pollen (Nebot et al. (2021) Cryopreservation and Freeze-Drying Protocols: 623-637). However, the published protocols use a fluidized bed dryer (FBD), which we demonstrate to be unsuitable for optimizing Poaceae pollen viability and longevity. In addition, these disclosed methods do not recognize the importance and interaction between the relative humidity (RH) or temperature for the dehydration of pollen, and how this affects the efficiency of fertility retention. The present invention improves significantly upon these methods.

The availability of preserved, viable Poaceae pollen overcomes many of the production challenges faced by the hybrid seed industry. With respect to hybrid seed, the availability of stored Poaceae pollen for delivery to female flowers can eliminate many standard, costly practices of seed production including, but not limited to, planting male plants separately from, but in proximity to, female plants to enable hybridization, isolation of female plants from undesired pollen sources, and use of genetic or mechanical male sterility of the female plants. These practices dramatically increase field space and resources dedicated to female plants which produce seed or grain. Eliminating any one of these practices would have an immediate positive impact on seed yield per acre. Moreover, stored pollen can be applied at any time. When pollen shed from male plants and pollen receptivity of female plants fail to coincide as planned (due to management, environment, or genetic variation), application of preserved, viable pollen ensures pollination of female plants at the optimal time. Pollination by undesired external (adventitious) sources of pollen or undesired self-pollination of female plants also can be reduced or eliminated by applying stored pollen of a desired type at the appropriate time. As such, methods of improving Poaceae pollen viability and extending the duration of its viability during and after storage are of significant value to the agricultural industry.

It is particularly important to note that although cryogenically stored Poaceae pollen may be capable of forming germination tubes, that does not necessarily indicate that the pollen also is capable of actually fertilizing an ovary, resulting in seed set. In many cases, cryogenically preserved pollen that forms a germination tube does not go on to fertilize the ovary and produce a seed, or it may not produce a viable seed. This invention demonstrates that by using the disclosed preservation and stabilization techniques, cryogenically stored pollen not only produces a pollen tube, but also results in actual seed set wherein said seeds are viable.

### BRIEF DESCRIPTIONS OF THE FIGURES

FIG. 1 shows the typical profile of conditions in a fluidized bed dryer (FBD) during Poaceae pollen dehydration. The RH in the tub increases as water is lost from the pollen and the temperature inside the tub declines due to evaporative cooling. Once most of the water is removed from the pollen, the RH and temperature in the tub return to their starting values.

FIG. 2 shows ears of corn resulting from pollinations using maize pollen subjected to three different drying treatments. The pollen in treatment A was dried for 30 hours in an RH box of specific interior conditions. The pollen in treatment C was kinetically dried in a fluidized bed dryer (FBD) to a PMC of 9.5%. The pollen in treatment B was kinetically dried in the same conditions as treatment C, and then transferred to an RH box of identical interior conditions to treatment A and held there for 29.5 hours. The pollen from each treatment was frozen at −80° C. after the drying treatments. The pollen from each treatment was subsequently later retrieved and used for pollinations with the kernel results shown in the photos.

FIG. 3 shows the number of minutes that pollen samples were dried in an FBD (x axis) plotted against the resulting number of kernels per ear (y axis) resulting from the pollinations. The graph demonstrates that the longer the time the pollen spent in the FBD, the lower its viability.

FIG. 4 shows the PMC as each pollen sample was removed from the FBD (x axis) as well as PMC after the RH box treatment (label next to each data point), while the y axis shows the number of kernels per ear resulting from the pollinations. Values show that following incubation in the RH boxes (and ahead of freezing), all samples had nearly identical PMCs (12.2-12.6%).

FIG. 5 shows the time that pollen samples were dried in the FBD (x axis) plotted against the average number of kernels per ear (y axis) resulting from subsequent pollinations using pollen from each treatment. FIG. 5 shows that there was a trend of reduced seed set with increasing time spent in the FBD.

FIG. 6 shows the mean number of kernels per ear for field pollinations (x axis) and greenhouse pollinations (y axis) conducted using pollen from 4 different males and 4 females. The pollen had been dried using an FBD and was subsequently frozen. Each point shows the average kernel number per ear for a specific male×female combination. Two values are missing from this chart because they were not recorded.

FIG. 7 shows ears of corn resulting from experiments in which pollen was dried in an RH box at either 5° C. for 73 hours (A), 12° C. for 46 hours (B) or 20° C. for 31 hours (C). Following drying, the pollen was placed into storage at −80° C. The pollen was subsequently used in pollinations. As seen from the representative ears from two reps, seed set was highest for treatment C and lowest for treatment A.

FIG. 8 shows the results of experiments using maize pollen dried at 2 different temperatures (5° C. and 20° C.) before freezing and later used for pollinations. The pollen was dried at the temperature and time shown on the x axis, until the noted PMC was reached, after which it was frozen at −80° C. The pollen from each treatment was subsequently later retrieved and used for pollinations with the kernel results shown on the y axis.

FIG. 9 is a graph plotting the PMC of dried pollen from eight field-based pollination experiments against the number of kernels obtained from using those dried pollen samples. The data was gathered from over 400 ears of corn pollinated by cryogenically preserved pollen. The pollen was gathered from four different males and was applied to ears of two different females. The figure demonstrates that the ideal PMC was determined to be very near to 15%.

FIG. 10 shows the PMC of dried pollen on the x axis and the average number of kernels generated by using the pollen on the y axis. The time to reach the PMC is provided in hours, next to each data point on the chart. The figure shows that the optimal PMC for cryogenically preserving statically dried pollen is roughly 12% to 15%.

FIG. 11 shows the VPD at the start of a pollen sample drying period on the x axis, while the number of kernels resulting from pollinations using the dried pollen samples following cryopreservation is on the y axis. The results show that a linear relationship exists between seed set on the ear and the VPD under which the pollen dried.

FIG. 12 shows representative ears of corn resulting from pollinations conducted over several years using a pollen bank of dried and cryogenically preserved pollen that was created in 2019. The pollinations were made on five occasions across various female hybrids and inbreds. Each pollen bank sample had a different pollen moisture content at the time of freezing. The results from these ears show that in general, pollen samples that were stored with lower PMCs tended to produce greater seed set.

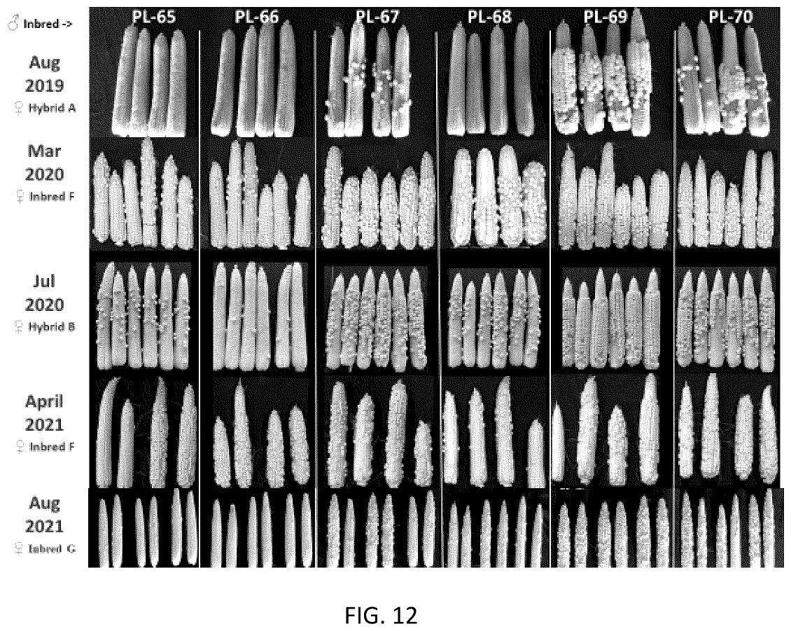

FIG. 13 is a line graph plotting the duration of pollinations conducted with a pollen gun on the x axis against the number of corn ears pollinated on the y axis. The figure demonstrates that approximately 10 ears could be pollinated per minute of pollen gun operation.

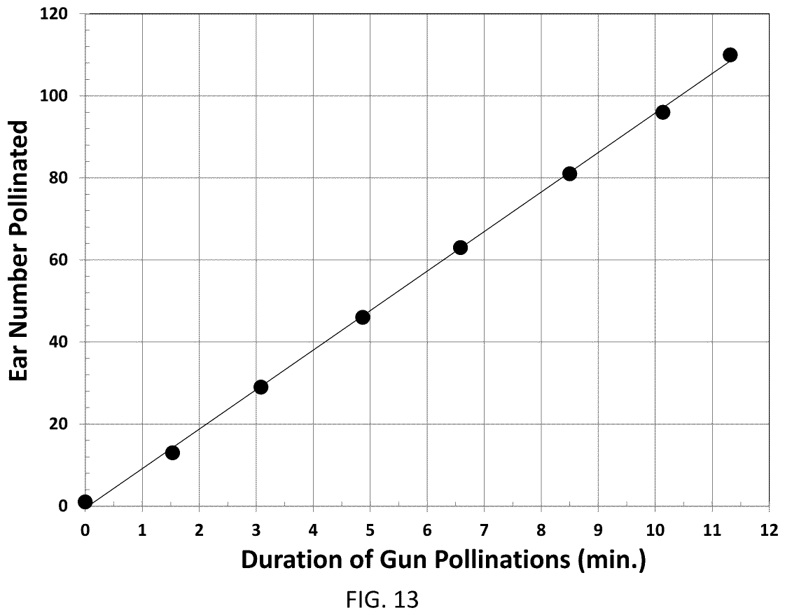

FIG. 14 is a graph that shows the order of pollination of a series of corn ears pollinated with a pollen gun on the x axis, plotted against the number of kernels counted on each ear on the y axis. This figure shows the performance of dried and cryogenically preserved pollen over a short period of time and emphasizes the importance of using a stabilization procedure following the removal of the pollen from cryogenic storage conditions.

FIG. 15 shows the average kernels per ear on the y axis for the pollinations conducted across three experiments described on the x axis. The pollinations included the three following treatments: machine-applied previously dehydrated and cryogenically stored pollen; hand-applied, metered doses of previously dehydrated and cryogenically stored pollen; and saturated doses of fresh pollen (also called pure saturated controls).

FIG. 16 shows the results of the application of pollen that has been dehydrated, cryogenically preserved, stabilized by thawing in controlled conditions, and then stored for different lengths of time. The kernels resulting from the pollinations are plotted on the y axis. The x axis shows the length of time each sample of pollen was stored after stabilization, and the temperature at which it was stored (room temperature (RT) or 5 degrees Celsius).

FIG. 17 plots the average number of kernels per ear on the y axis for several samples of pollen that were stored at 5 degrees Celsius. The number of days for which each sample was stored is shown on the x axis. The female that was pollinated as well as the date are also shown below the x axis. One line on the graph is for dried pollen that was dried to a PMC of 13.6%, while the second line is for pollen that was not dried but otherwise maintained and used in the same way as the dried pollen.

FIG. 18 shows the outcome of a 3×3 factorial study. The y axis shows the normalized number of kernels per ear, while the x axis shows the temperature in degrees Celsius. Each RH treatment described in working example 16 generates a separate seed set curve, indicative of pollen viability. The calculated equation for each curve has been plotted for each of the treatments and the equation for each curve is extended to show the anticipated results of drying at lower temperatures.

### SUMMARY OF THE INVENTION

Provided are methods of preserving Poaceae pollen for use in pollinating and fertilizing Poaceae plants comprising drying said pollen in controlled conditions wherein the movement of the pollen is minimized and then storing said pollen, such that at least a portion of the pollen remains viable during and after the storage so it can be used to successfully pollinate and fertilize Poaceae plants. In some embodiments the storage is conducted below 0 degrees Celsius, while in other embodiments, the storage is conducted below −20 degrees Celsius or at about −80 degrees Celsius. In yet another embodiment, the storage is conducted at temperatures between 0 and 10 degrees Celsius, or above 10 degrees Celsius.

Also provided are methods of drying or dehydrating pollen wherein during the drying step, the pollen is gently moved to prevent aggregation of pollen grains. In some embodiments, the pollen is dried to a pollen moisture content ranging from 10% to 22%, while in other embodiments the pollen is dried to a pollen moisture content of about 15%. The rate at which said pollen is dried is influenced by one or more of the following parameters: relative humidity, vapor pressure deficit, airflow, the amount of pollen being dried, the starting pollen moisture content, radiant energy, and temperature. In some embodiments, one or more of the previously listed parameters is adjustable. For example, in some embodiments, the temperature ranges from 5 degrees Celsius to 30 degrees Celsius, or from 15 degrees Celsius to 25 degrees Celsius, or from 24 degrees Celsius to 25 degrees Celsius. In other embodiments, the vapor pressure deficit ranges from 0.1 kPa to 2.0 kPa.

Further provided are methods of applying the stored pollen to the stigmas of a species of Poaceae plant capable of pollination and fertilization by the stored species of Poaceae pollen. Prior to the application of said pollen, it can optionally be stabilized. The stabilization methods encompass thawing the pollen, such that the full volume of frozen pollen is quickly thawed. In some embodiments, the full volume of frozen pollen is thawed within a period ranging from 10 seconds to 300 seconds. The methods for thawing and stabilizing pollen include exposing the frozen pollen to a temperature ranging from 36 degrees to 44 degrees Celsius, such as in a water bath wherein the water is at a temperature ranging from 37 degrees to 42 degrees Celsius.

The methods provide for the application of the thawed, stabilized pollen to the stigmas of a species of Poaceae plant capable of pollination and fertilization by the species of thawed Poaceae pollen. In some embodiments, the thawed pollen is further stabilized by storing it at a temperature above 0 degrees Celsius for up to twenty-four hours before application to said Poaceae plant. In other embodiments, the storage of the thawed pollen occurs at a temperature ranging from 1 degree Celsius to 23 degrees Celsius.

The provided methods of applying thawed pollen to stigmas of receptive plants are intended to result in Poaceae seeds, which can optionally be intended for seed production purposes. In some embodiments, the amount of thawed, and optionally stabilized, pollen applied to the Poaceae plant results in about 31 to 100% of the number of seeds that would have resulted from application of the same amount of fresh pollen, or about 50 to 100% of the number of seeds that would have resulted from application of the same amount of fresh pollen, or about 50-90% of the number of seeds that would have resulted from application of the same amount of fresh pollen.

In some embodiments, the drying or dehydration of the pollen occurs in a relative humidity box. The methods provide for drying and dehydration of pollen during which the pollen movement is minimized, which thereby reduces damage to the Poaceae pollen in comparison to pollen preservation methods that do not minimize pollen movement. The methods provide for the control of pollen moisture removal to result in a predetermined rate of drying.

Also provided by the invention is a method of stabilizing cryogenically stored pollen comprising drying or dehydrating said pollen in controlled conditions wherein the movement of the pollen is minimized, subjecting the pollen to cryogenic storage conditions, rapidly thawing the Poaceae pollen such that the temperature of the pollen exceeds 0 degrees Celsius, and then stabilizing the thawed Poaceae pollen by maintaining it at a temperature above 0 degrees Celsius and below 40 degrees Celsius, wherein the pollen grains maintain viability such that they can be used to successfully pollinate and fertilize the species of Poaceae plant from which the pollen was collected. The thawing of the pollen in this method can be conducted, for example, in a water bath, including a water bath at a temperature ranging from 36 degrees Celsius to 44 degrees Celsius.

Also provided by the invention is a composition comprising Poaceae pollen that has been dried in conditions wherein the movement of said pollen is minimized, which is subsequently stored at a temperature such that at least a portion of said pollen remains viable during and after said storage and can be used to successfully pollinate and fertilize Poaceae plants. In some embodiments the composition is stored below 0 degrees Celsius, while in other embodiments, the composition is stored below −20 degrees Celsius or at about −80 degrees Celsius. In yet another embodiment, the composition is stored at temperatures between 0 and 10 degrees Celsius, or above 10 degrees Celsius.

Furthermore, the composition may be dried to a pollen moisture content ranging from 10% to 22%, including a pollen moisture content of about 15%. The invention includes methods of applying the composition to the stigmas of a species of Poaceae plant capable of pollination and fertilization by the species of Poaceae pollen contained in the composition. The composition may also be stored for up to twenty-four hours at a temperature above 0 degrees Celsius before application to said Poaceae plant. The method encompasses the application of the composition to a Poaceae plant such that said application results in Poaceae seeds.

In addition, the composition may be stabilized by drying or dehydrating said pollen in controlled conditions wherein the movement of the pollen is minimized, subjecting the pollen to cryogenic storage conditions, rapidly thawing the Poaceae pollen such that the temperature of the pollen exceeds 0 degrees Celsius, and then stabilizing the thawed Poaceae pollen by maintaining it at a temperature above 0 degrees Celsius and below 40 degrees Celsius, wherein the pollen grains maintain viability such that they can be used to successfully pollinate and fertilize the species of Poaceae plant from which the pollen was collected.

### DETAILED DESCRIPTION

The following is a detailed description of an embodiment of technology and methods enabling improved long-term storage and stabilization of collected Poaceae pollen. The pollen may be collected from actively shedding plants or, alternatively, the pollen may have been previously collected and stored according to numerous methods known in the art that maintain pollen viability over a period of time. Such methods include, for example, freezing, freeze-drying, storing in liquid nitrogen, etc.

For the purposes of this disclosure, the term “viable” or “viability” is used to describe Poaceae pollen that is able to germinate and grow a pollen tube to at least a length twice the diameter of the pollen grain. In addition, Poaceae pollen can be judged viable by demonstration that the cellular nature of the material remains integral and is judged to maintain intactness such that normal cellular processes of metabolism and intracellular functioning is possible. The viability of Poaceae pollen can be assessed in numerous ways, including, but not limited to, assessment of pollen tube growth on artificial media or excised stigmas or styles, assessment of cellular intactness by vital staining of numerous sorts, absence of electrolyte (e.g., potassium) leakage, and impedance flow cytometry. Viable Poaceae pollen can successfully germinate and commonly possesses the vigor necessary to promote fertilization and initiation of seed development. Not all viable pollen is also fertile pollen. In some cases, even when a pollen grain is viable and commences with pollen tube growth, it may lack the vigor necessary to reach the ovule and promote fertilization. Non-viable pollen grains cannot successfully germinate. Viability can refer to a single Poaceae pollen grain or a population of Poaceae pollen grains. When a percentage value is used to describe pollen viability, the value is typically being applied to a population of pollen. For the purposes of this disclosure, the term “successfully pollinate” means that the Poaceae pollen is viable and is able to germinate and fertilize a plant such that the plant produces seed in response to the pollination.

Another term that can be used to describe the ability of pollen to germinate and form the pollen tube is “germinability.” Thus, Poaceae pollen with good viability is pollen that is desirable for use with the methods in the present disclosure. The “viability window” refers to the limited lifespan during which pollen remains viable.

For the purposes of this disclosure, the term “fertile” or “fertility” is used to describe the ability of Poaceae pollen to deliver the sperm nuclei to the ovule and thereby effect double fertilization. In flowering plants, the term “double fertilization” refers to one sperm nucleus fusing with the polar nuclei to produce the endosperm tissues, and the other sperm nucleus fusing with the egg nucleus to produce the embryo.

For the purposes of this disclosure, “loss of viability” and “loss of fertility” are terms used to describe Poaceae pollen. These terms mean, respectively, that the viability and fertility of the pollen has fallen to a level below that required for successful initiation of seed development. The level of viability and fertility required for successful pollinations can be determined for different plants. In maize, for example, the viability and fertility required for successful pollinations is typically defined to be an average of 4 grains of fresh pollen per ovule, or 4 to 20 grains of preserved pollen (Westgate et al. (2003). Crop Science 43:934-942). Preserved pollen includes pollen which has been preserved according to the methods of the present disclosure. Pollen preserved using the methods of the present invention may require fewer grains for successful maize pollination, such as 4 to 13 grains of preserved pollen.

For the purposes of this disclosure, the term “longevity” is used to describe the length of time that Poaceae pollen remains both viable and fertile.

For the purposes of this disclosure, the term “storage” means any period of Poaceae pollen containment with the intent of using the pollen at a later time or date. The term “preservation” means any storage of Poaceae pollen that results in a level of viability, fertility, or both, which is different than the level of viability, fertility or both, which would occur if the pollen were held in unregulated environmental conditions.

For the purposes of this disclosure, the term “female plant” is used to mean a plant that is being used as the recipient of the Poaceae pollen, and which has receptive flowers that are being pollinated and subsequently fertilized. In the case of maize, and many other species, the plant is monoecious and contains male and female inflorescences on a single plant. In the practice of breeding, pollination, cross-pollination, and hybridization, some plants act as the “male parent plant” from which pollen is collected for use in pollinations, and some plants act as the “female parent plant” being the recipient of the pollen. In the case of self-pollinations, a single plant is acting as both the “male parent plant” and the “female parent plant” because the female flowers are fertilized by pollen from its own male flowers.

For the purposes of this disclosure, the term “fresh” when applied to Poaceae pollen means pollen released from the anthers of a flower which, in its natural pattern of organ growth and development, releases pollen upon anther dehiscence in response to promotive environmental conditions.

The collection of fresh Poaceae pollen may be conducted in ways commonly known in the art. For example, pollen may be collected from freshly shedding flowers or male flower structures produced in any variety of manners. In the case of maize, for example, pollen is collected from freshly-shedding anthers of male flowers borne on tassels, which may be attached, or detached, from the plant. The pollen may be collected from plants grown in any environment suitable for plant growth. Such environments include, but are not limited to, a field, a growth chamber, a greenhouse, a glasshouse, a shade house, a hoop house, a vertical farming facility or a hydroponic facility. Alternatively, Poaceae pollen may be collected directly from anthers by crushing or grinding the anthers, thereby releasing the pollen and allowing for its collection. In addition, pollen may be collected from a tassel culture facility (Pareddy D R, Greyson R I, Walden D B (1989) Theor Appl Genet 77:521-526). Cultured tassels may be mature tassels that have been removed from plants in any type of growing facility or environment, including a field or other type of growing facilities, and placed into water in a controlled environment to collect pollen. Alternatively, cultured tassels may also be tissue that has been harvested from flowering structures at immature stages and then cultured to develop into a tassel. The collected Poaceae pollen may have been stored for a period of time before subjecting it to the preservation or stabilization procedures described in this disclosure.

A critical factor in maintaining Poaceae pollen viability, regardless of the temperature at which the pollen will be stored, is a very controlled drying process (also called a dehydrating process), which cannot practically be achieved in the field and is absent from the currently disclosed methods for pollen preservation. The controlled drying process includes the control of the RH, the VPD, and the temperature at which the drying takes place. The length of time required to dry the pollen is a function of the correct combination of RH, temperature, and pollen-derived water to be removed. Research on this invention has demonstrated that the drying rate can be altered in order to predict the pollen moisture content, and therefore the viability and fertility of the pollen. In addition, the evaporative demand is an important factor when preparing pollen for storage. The lower the VPD at the onset of storage, the higher the fertility of the pollen after storage. On occasion, some pollen may actually need to be hydrated rather than dried before it can be stored, but this is relatively rare because pollen naturally has a relatively high moisture content.

In order to preserve the collected Poaceae pollen, regardless of whether it is fresh or previously preserved, it must have a suitable pollen moisture content. In most cases, the pollen must be dried (dehydrated) before it can be preserved. Drying the pollen takes place in conditions wherein the pollen remains still and where movement is minimized. The drying of Poaceae pollen may be accomplished and/or controlled by regulating the RH to which the pollen is exposed. In order to achieve a known RH level, a variety of means known in the art may be employed. These include, but are not limited to, using an apparatus such as a dew-point generator, an atomizer, a mixed-flow generator, a sonicator, or other apparatus designed to increase RH. It can also be achieved using a two-pressure process, a two-temperature process, or a saturated salt solution.

A saturated salt solution is a mixture of salt and water that contains sufficient salt to exceed the solubility of said salt in water. The interaction of the salt and water reduces the water vapor concentration and the relative humidity over the solution, compared to pure water. For example, a saturated salt solution of LiCl will generate a relative humidity over the solution of 11 percent, where a saturated salt solution of KCl will generate a relative humidity over the solution of 85 percent. The exact values may vary slightly depending on temperature and purity of the salt and water. The RH value generated by the salt solution is the important factor in the practice of the invention. The chemical nature of the salt that is used is not critical as long as it provides the correct RH value.

The two-pressure humidity generation process involves saturating air or nitrogen with water vapor at a known temperature and pressure. The saturated high-pressure air flows from the saturator, through a pressure reducing valve, where the air is isothermally reduced to test pressure at test temperature. Likewise, the two-temperature method circulates an air stream through a precise temperature-controlled saturator (water spray or bubble column). The air becomes saturated at the temperature of the water. When leaving the saturator, the air travels through a mist elimination device to ensure liquid water does not go beyond the saturator. The air is then reheated to the desired dry bulb temperature. The temperature of the saturator would equal the dew point temperature. RH is calculated from the dew point and dry bulb temperatures (two temperature method).

In most cases, it is initially necessary to remove some moisture from the Poaceae pollen in order to maximize the pollen's viability and fertility, and the removal of moisture is particularly critical prior to long-term cryogenic storage. As noted below, to maintain the viability and fertility of the pollen, the moisture removal may be regulated. The pollen moisture removal may be influenced by many factors. These factors include, but are not limited to, air movement (also called airflow), vapor pressure deficit, RH, radiant energy, temperature, the amount of pollen being dried, and the starting pollen moisture content. Airflow includes the disruption of a boundary layer of air between said pollen grains and the air near said pollen grains. The boundary layer between a solid surface and the atmosphere around it is a thin zone of calm air next to the surface through which gasses and energy are exchanged between the surface and the surrounding air. A thin layer of pollen in a drying chamber, for example, forms a moderately rough surface that will form a boundary layer of varying thickness depending on the turbulence of the air moving over the pollen. The thickness of the boundary layer influences how quickly gases and energy are exchanged between the pollen and the surrounding air. A thick boundary layer reduces the transfer of heat, water vapor, and other gasses from the pollen to the environment. (gpnmag.com/article/the-boundary-layer-and-its-importance). Airflow over the surface decreases the boundary layer to promote vapor and energy exchange between the pollen and the atmosphere.

Referring to RH, establishing an environment with a known RH may serve to gradually remove moisture from the pollen, thereby acting as a drying agent. In some cases, when pollen is very dry and needs to be hydrated, the environment with the known RH can also serve to hydrate pollen grains. Because RH is only one factor, a wide range of RH values may be used based on the interaction of RH with other factors influencing the total pollen moisture loss. As such, depending on whether the pollen requires dehydration or hydration, the RH may range from 0-100%, or 10-85%, or 75-85%. Accordingly, the RH may be about 10%, about 15%, about 20%, about 25%, about 30%, about 35%, about 40%, about 45%, about 50%, about 55%, about 60%, about 65%, about 70%, about 75%, about 80%, about 85%, or higher. Moreover, the RH may be adjustable over the period of dehydrating or hydrating. As RH rises, VPD decreases, which results in a lower pollen moisture loss. On the other hand, as RH decreases, VPD increases, which can lead to a greater pollen moisture loss.

In general, during dehydration, the temperature should be at least 1 degree Celsius higher than the ambient temperature of the air in the environment or chamber in which the pollen is being maintained. In all cases, the temperature should be above 0 degrees Celsius to prevent the pollen from freezing. If a rapid rate of drying is desired, a higher temperature can be used with a lower RH. Conversely, if a slower rate of drying is desired, a lower temperature can be used combined with a higher RH. The volume of pollen to be dried may be an influencing factor on the choice of temperature. One of skill in the art will understand that the temperature used for any given pollen dehydration can vary depending on the desired speed of drying. The effect of temperature on the dehydration curve is explored in more detail in working example 16 of this disclosure.

Accordingly, the lower the RH level threshold, the faster moisture is removed from the pollen. The moisture content of fully hydrated Poaceae pollen is typically about 60%. A target PMC prior to cryogenic, refrigerated, or ambient temperature storage is a value in the range of about 10% to about 30%, including about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, and about 10% moisture content. In some embodiments, the target moisture content of the pollen prior to cryogenic, refrigerated, or ambient temperature storage is about 10-22%, and in other embodiments is about 15%. The RH can be adjusted over time to allow the rate of drying to increase or decrease.

The physico-chemical process of drying high moisture pollen to specific moisture contents relies on the controlled removal of water in the liquid state within the pollen grain to the atmosphere surrounding the pollen in the vapor state. The driving force controlling this drying process depends on the difference in energy status of the ‘free water’ within the pollen grain (i.e., water that can exchange with the environment) and the energy status of the water in the atmosphere. This difference in water energy status can be described in several ways but is most often quantified as the VPD between the vapor pressure of the water in the pollen and the vapor pressure of the water in the atmosphere. Once pollen is released from anthers in the field, the driving force for water vapor transfer from the pollen to the atmosphere is extreme because the vapor pressure of the air is typically 1000 times less than that of the water within the pollen grain. The pollen dries very quickly. Controlled drying, however, can be achieved by regulating the VPD around the pollen grains. Maize pollen, for example, is released from the plant at about 60% moisture, by weight. This moisture content corresponds to an energy status of about −20 MPa, equivalent to approximately 98% saturation, or 98% Relative Humidity (Westgate, M. E. and Boyer, J. S. 1986. Crop Sci. 26:947 951). As such, pollen drying and rehydration can be purposefully controlled by manipulating the temperature and RH (i.e., the VPD) of the atmosphere of a chamber containing the pollen. The rate of drying or rehydration then is a function of the volume of pollen within the chamber and the VPD of the atmosphere around the pollen.

In general, the vapor pressure of the ambient air in which the pollen is being maintained should be at least 0.1 kPa lower than the effective vapour pressure of the moisture in the pollen being dehydrated, unless the pollen requires hydration, in which case the vapor pressure of the ambient air in which the pollen is being maintained should be at least 0.1 kPa higher than the effective vapor pressure of the moisture in the pollen being hydrated.

In some cases it may be necessary to subject pollen to a hydrating step, instead of a dehydrating step. In such cases, the pollen can be exposed to an environment in which the vapor pressure of the ambient air in which the pollen is being maintained is at least 0.1 kPa higher than the effective vapor pressure of the moisture in the pollen being hydrated. The pollen moisture content can be increased by flowing one or more gases into the environment surrounding the pollen wherein the gases have a specified RH. Alternatively, the chamber or environment in which the pollen is being stored or maintained can be subjected to other well-known means of increasing the RH as described earlier in this disclosure. In this embodiment, the pollen moisture content can be increased in a slow and controlled manner, such that a target PMC is attained.

It is important to prevent physical stress to the Poaceae pollen during handling and particularly during the dehydration process. In particular, work to support this invention has identified that Poaceae pollen which remains still during drying (referred to as “passive drying” or “static drying”) in preparation for storage has a higher fertility level when compared to pollen which is dried in a system with active movement of the pollen (referred to as “kinetic drying”). In a kinetic drying environment, the pollen contained within the drying apparatus is agitated mechanically, such as by means of vibration, forced air, rotation, or other means. Even though, in many cases, the movement experienced by the pollen grains is relatively small, the agitation still has an adverse effect on the viability and longevity of the pollen grains. While the agitation serves to expose the maximum surface area of the pollen to the air, thereby providing potential for more even drying and preventing aggregation of the pollen, kinetic drying may be disruptive to the pollen plasma membrane and endomembrane system integrity, at least in part as a result of the friction experienced during the kinetic drying process. Accordingly, practice of the invention uses methods that dry the Poaceae pollen without movement, or such that the movement is so small that it does not adversely affect the health, longevity, or viability of the pollen grains. Such methods of drying minimize motion of the pollen grains and minimize friction between the surfaces of pollen grains or between a pollen grain and any other surface.

In some cases, it is necessary to gently move the Poaceae pollen to prevent aggregation (i.e., clumping) of pollen grains. During the drying process, particularly when there is a thick layer of pollen being dried, the pollen grains tend to aggregate, which prevents the even drying of the pollen grains. In order to achieve even drying and to prevent further aggregation, the pollen grains can be gently moved, such as by stirring the grains, applying gentle pressure to aggregations of pollen, turning over the grains, or otherwise disrupting the aggregations in such a way as to minimize movement and agitation of pollen grains and to minimize any damage to the pollen plasma membrane or endomembrane system. This process of occasionally disrupting aggregations of pollen does not have an adverse effect on the overall health and viability of the grains. The disruption of the aggregations can be achieved manually or mechanically. Because it is brief and limited, this kind of movement of the grains is distinctly different from a general sustained movement and agitation of the grains such as would be associated with the use of a fluidized bed dryer or other kinetic drying methods.

Once the Poaceae pollen has reached the optimal PMC of 11% to 30%, it can be stored. Storage of pollen may be achieved by placing the pollen into a container or chamber where environmental conditions are regulated. For example, a refrigeration chamber with controlled temperature and the ability to control RH could be used to preserve the pollen. Likewise, a room suitable for storing larger volumes of pollen, which is supplied by a mechanical form of humidification (sonic, ionized, etc.), dehumidification, and is temperature controlled may be used. Pollen may be placed into suitable containers and frozen, refrigerated, or maintained at ambient temperatures for a period of time.

Storage can be achieved by placing the pollen in liquid nitrogen, in freezing conditions of −80° C. to 0° C., in refrigeration, or at atmospheric conditions. The choice of conditions in which to store the pollen may depend upon the timeframe in which the pollen is expected to be used.

Poaceae pollen subjected to the preservation method and storage method described herein may be used in any application where pollen is a commercial or experimental unit. In addition, the pollen may be subjected to further stabilization as described later in this disclosure. In one embodiment, the preserved pollen may be used to produce seed, hybrid, parent, or otherwise, in any setting, including but not limited to a laboratory, greenhouse, and field. In another embodiment, the preserved pollen may be used to produce grain, hybrid or otherwise, in any setting, including but not limited to a laboratory, greenhouse, and field.

The preservation techniques disclosed in this invention are intended to successfully treat and preserve pollen such that the preserved pollen maintains its viability. Post-preservation viability can be expressed in different ways. For example, the pollen preserved in accordance with this invention maintains its viability to the extent that about 4 to about 20 grains of pollen are sufficient to successfully pollinate an ovule. Alternatively, the viability of the pollen following preservation can be measured as a percentage. In some embodiments, at least 90% of the pollen remains viable after preservation, such that at least 90% of the pollen grains are able to successfully pollinate and fertilize a Poaceae plant of the same species. In some embodiments, at least 90%, at least 91%, at least 92%, at least 93%, at least 94%, at least 95%, at least 96%, at least 97%, at least 98%, at least 99%, or 100% of the pollen grains may retain viability. The pollen of some Poaceae species is more difficult to preserve than others. Accordingly, in some embodiments, at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, or at least 89% of the pollen grains may retain viability.

Research conducted in support of this application has demonstrated that Poaceae pollen that has been dried in controlled conditions as described in this disclosure and then stored at temperatures below freezing can be thawed and stabilized in controlled conditions. The stabilization process reacclimates the pollen following freezing by rapidly thawing it and maintaining in controlled conditions to stabilize it such that it will have extended viability compared to pollen that is not subjected to such conditions. The methods herein disclosed of stabilizing pollen after drying and cryogenic storage have a significant impact on its viability. Experimentation has shown that an extremely rapid thaw is superior to any form of slow thawing, because rapid thawing promotes a rapid transition from ice to liquid and prevents undesirable refreezing, all of which prevent the likelihood of internal damage in the pollen grain. Thawing previously dehydrated and cryogenically stored Poaceae pollen must be conducted in a manner that keeps the pollen plasma membrane and endomembrane system intact and that retains the viability of the pollen such that it can be used to successfully pollinate and fertilize the Poaceae species from which the pollen was collected.

In particular, after storage, pollen that has been thawed tends to lose viability quickly, such that it can no longer be successfully used to pollinate and fertilize plants. For example, in working example 12 of this disclosure, it is demonstrated that thawed maize pollen that is not subjected to stabilization is only viable for about 10 minutes. Pollen that has been cryogenically preserved using the techniques described in this invention that is subsequently warmed using the techniques described herein is then more stable than any previously disclosed preservation and reacclimation method for Poaceae pollen. As described in Example 14, the pollen can even be used 48 hours later to successfully pollinate maize plants and result in seed set. This is a significant advancement in the field of pollen preservation and stabilization.

The initial step in the stabilization of previously dehydrated and cryogenically stored pollen requires that essentially the entire portion of pollen being stabilized reaches the same temperature at the same time. The rate of thaw is the critical element in the process, because the thawing must be achieved efficiently and in a manner that avoids refreezing during the thawing process across the entire pollen portion being stabilized, regardless of the pollen volume. Accordingly, the temperature of the stabilization process must always remain above 0 degrees Celsius. The frozen pollen must be brought to ambient temperature as rapidly as possible without damaging the integrity of the pollen plasma membrane or endomembrane system. One of skill in the art will understand that various warming systems can be used to achieve this objective. For example, in one embodiment, the pollen is subjected to a constant temperature ranging from about 36 to about 44 degrees Celsius. In another embodiment, a portion of previously frozen pollen is placed in a container that is then exposed to a warm water bath, such that the entire portion of pollen is brought to the same temperature within a few minutes. The warming mechanism must be consistent and must warm the pollen portion consistently throughout the volume of pollen being warmed. In many cases, the warming is completed within seconds, such as within 10 seconds, within 20 seconds, within 30 seconds, within 40 seconds, within 50 seconds, or within 60 seconds. In some cases, the warming can take longer, such as from 60 seconds to 100 seconds, from 101 seconds to 150 seconds, from 151 seconds to 200 seconds, from 201 seconds to 250 seconds, or from 251 seconds to 300 seconds. Depending on the warming mechanism being used and the volume of pollen, the warming may require more than 5 minutes, and may take up to 10 minutes, up to 20 minutes, up to 30 minutes, up to 40 minutes, up to 50 minutes, or up to 60 minutes.

Although various representative embodiments of this invention have been described above with a certain degree of particularity, those skilled in the art could make numerous alterations to the disclosed embodiments without departing from the spirit or scope of the inventive subject matter set forth in the specification and claims. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements, and/or substantial equivalents.

The following examples illustrate the present invention in more detail and are illustrative of how the invention described herein has been demonstrated in maize.

### Example 1: Drying Pollen with a Fluidized Bed Dryer

In 2019, research was conducted using kinetic drying through the use of a fluidized bed dryer (FBD) to dehydrate pollen in preparation for freezing. In an FBD, pollen is “floated” in a stream of air. The air moves past the pollen grains in this form of kinetic drying, which can be controlled for flow rate, temperature and/or RH, all of which can affect the rate of pollen dehydration. Employing kinetic drying can cause variable stress to the fertility of pollen through rapid physical, and presumably frictional, forces.

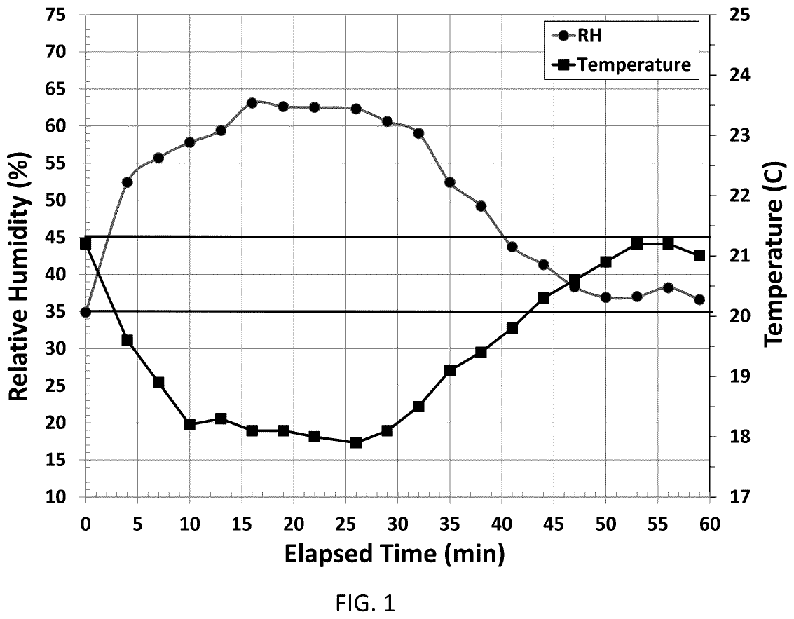

In a representative experiment to test the suitability of an FBD for drying pollen, maize pollen was placed in a Sherwood M501 FBD, where it floated and dried in a glass container called a tub. FIG. 1 shows the typical profile of tub conditions during dehydration. Specifically, in a typical drying process of maize pollen, the RH in the tub increases as water is lost from the pollen and the temperature inside the tub declines due to evaporative cooling. Once most of the water is removed from the pollen, the RH and temperature in the tub return to their starting values. It was determined that charts of the type shown in FIG. 1, which show the change in RH and temperature during pollen drying, can be used to pinpoint a time when the drying process can be halted in order to obtain pollen of a specific pollen moisture content (“PMC”).

Other experiments were conducted in which drying was controlled to a specific PMC by simply weighing the pollen and tub at the start of the drying process and monitoring, during drying, the change in sum weight at periodic time points. Weight change is attributed to water loss and, in turn, real-time changes in PMC can be calculated.

### Example 2: Assessing the Fertility of Kinetically Dried and Passively Dried Pollen

In this experiment, the fertility of maize pollen that was dried in 2 different ways was assessed. The first drying method was the kinetic method as described in Example 1. The second drying method used a relative humidity (RH) box. In an RH box, saturated salt solution is placed in a sealed container (plastic, metal, or glass) and fresh pollen is placed on a screen platform above the solution. A particular RH forms in the container on the basis of the salt chosen for use. Furthermore, a specific vapor pressure deficit (VPD) forms in the container as a function of the RH produced and the temperature at which the container is incubated. The pollen dehydrates according to the VPD in the container. Small fans or air pumps are also used with the container to cause interior air movement (mixing) to various degrees. This can disrupt the boundary layer naturally formed at the surface of the pollen and affect the speed of pollen dehydration. Pollen in this system remains stationary during dehydration. Pollen dried in RH-boxes is essentially motionless and experiences no frictional forces.

A portion of fresh pollen was kinetically dried in a FBD to a PMC of 9.5% in about 30 minutes. The FBD was operated with an average fan speed setting of “13” (max setting=100). Some of the dried pollen was immediately frozen at −80° C., while another share of the kinetically dried pollen was transferred to an RH box of specific interior conditions and held there for 29.5 hours.

A second portion of the fresh pollen was dried for 30 hours in a duplicate RH box of identical interior conditions. Pollen from both RH box treatments, held for 29.5 or 30 hours, was then frozen at −80° C. and later retrieved for use in pollinations.

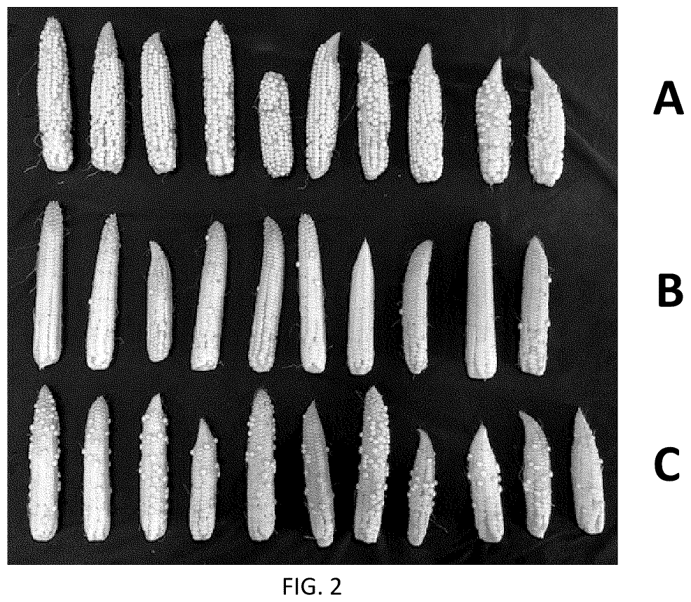

Accordingly, the maize pollen was dried in three ways: “FBD only,” dried in the FBD ahead of further conditioning in a RH box, or dried using a RH box only. The results of the experiment are shown in Table 1. Corn ears with seed produced from pollinations conducted with the pollen subjected to these treatments are shown in FIG. 2

The results show that seed set was sharply reduced by drying in the FBD and subsequent conditioning in the RH box did not reverse this situation. In contrast, pollen dehydration employing only static drying in a RH box resulted in pollen that (following freezing and subsequent use in pollinations) could form ample seed. The PMC of the pollen dried only with the FBD (kinetically) is significantly lower than pollen dried passively and this could be a contributing cause of reduced seed set.

TABLE 1
Seed Set Comparison for
Statically and Passively Dried Pollen

| | | Mean + |
|---|---|---|
| | PMC | SE |
| | After | Kernels/ |
| Treatment | Drying | Ear |
| A: Static Drying Only (RH Box) | 11.8 | 249 ± 24 |
| B: Kinetic Drying followed by Static Drying | 12.0 | 2 ± 1 |
| C: Kinetic Drying Only (FBD) | 9.5 | 34 ± 7 |

### Example 3: Varying the Duration of FBD Drying Time

Experiments were conducted to assess how the length of drying time in the FBD impacted pollen fertility. Fresh maize pollen was placed in an FBD for dehydration. The FBD was operated with a fan speed setting of “12” (max setting=100). Pollen was removed from the FBD after various durations of kinetic drying. Samples were dried in the FBD for 0, 5, 10, 15, 20, or 25 minutes and then transferred to one of several RH boxes. All RH boxes had nearly identical interior RH and temperature conditions for static, motionless drying. All pollen samples were removed from the RH boxes after 32 hours of incubation and frozen at −80° C. Pollen samples were later removed from the −80° C. freezer and used in pollinations.

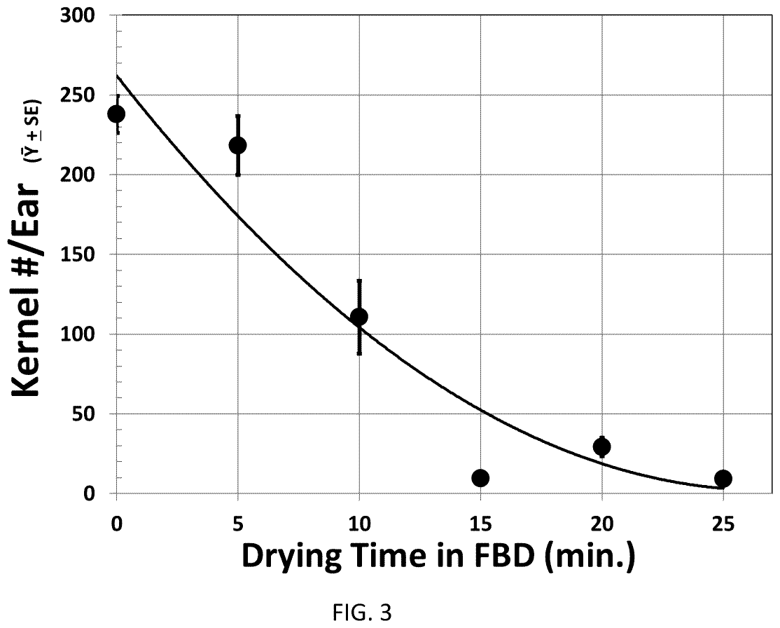

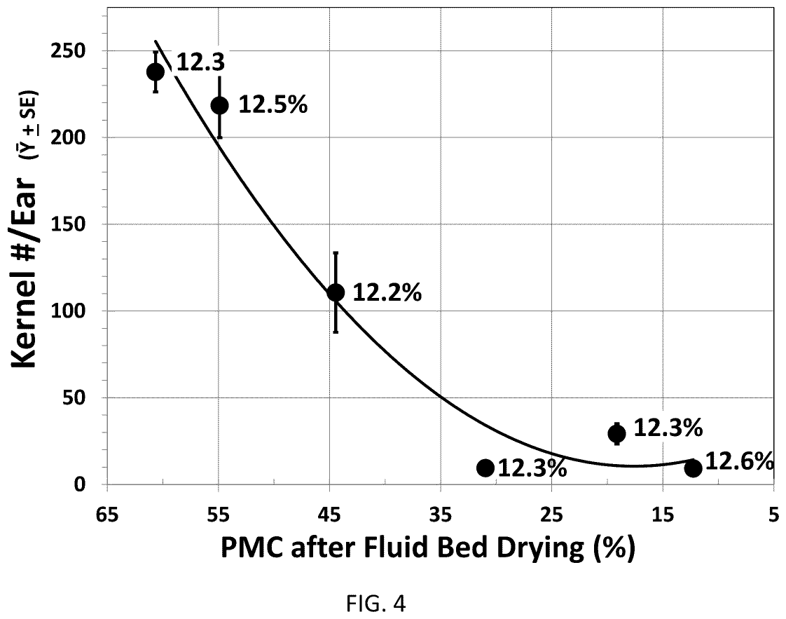

FIG. 3 shows the number of minutes that the pollen samples were dried in the FBD plotted against the resulting number of kernels per ear from the pollinations. FIG. 4 shows the PMC as each sample was removed from the FBD (x axis) as well as PMC after the RH box (values next to each data point), while the y axis shows the number of kernels per ear resulting from the pollinations. Values indicated on FIG. 4 show that following incubation in the RH boxes (and ahead of freezing), all samples had nearly identical PMCs (12.2-12.6%). FIGS. 3 and 4 together demonstrate that the longer the pollen was kinetically dried, the lower the resulting seed set, regardless of the fact that all pollen samples had very similar PMC values ahead of freezing.

### Example 4: Minimizing FBD Fan Speed

To analyze whether minimizing the fan speed of the FBD would improve pollen viability, fresh maize pollen samples were dried in an FBD for times ranging from 0 to 50 minutes and then a subsample of pollen from each drying time treatment was transferred to an RH box to complete drying. The initial drying was performed with an operating fan speed setting of “3,” which is the slowest fan speed possible on the Sherwood M501 FBD. Subsamples of pollen transferred from the FBD to an RH box were held in the RH boxes for 41 hours. All RH boxes had nearly identical interior RH and temperature conditions for static, motionless drying. Following removal from the RH boxes and ahead of freezing, pollen samples had a PMC ranging from 11.8% to 12.5%. Because of the slow fan speed, the pollen in the FBD did not actually “float” when the fan was operated, and instead the pollen mass had the appearance of a “gentle rolling boil.” Following freezing, pollen from each treatment was subsequently used in pollinations and the number of kernels resulting from the pollinations was determined.

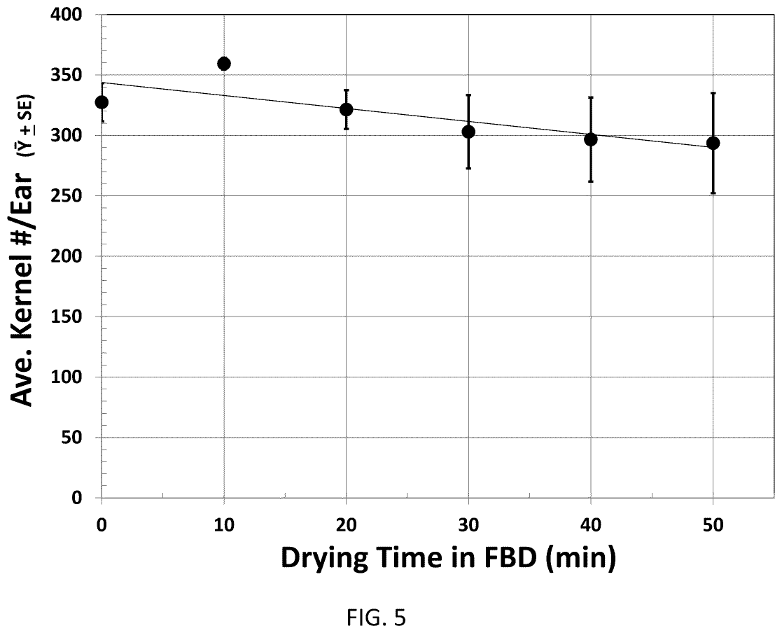

The results of this experiment are shown in FIG. 5, which plots the drying time for each sample of pollen in the FBD (x axis) against the average number of kernels per ear (y axis) resulting from the subsequent pollinations. Despite pollen samples experiencing minimal physical agitation in the FBD, and having very similar PMCs in ultra-cold storage, when used in pollinations, FIG. 5 shows that there was a trend of reduced seed set with the increasing time the pollen was kept in the FBD. This result further confirms the importance of maintaining the pollen in a still condition during drying with minimal agitation or friction.

### Example 5: Using Dried and Cryogenically Preserved Pollen to Pollinate Different Maize Inbreds

In 2020, an experiment was conducted to see how well the dried and cryogenically preserved maize pollen worked when using pollen from four different inbreds (i.e., 4 males). Pollen for this experiment was dried kinetically in an FBD, using average fan speed settings of 16, 15, 12, and 19 for males 1-4, respectively. The dried pollen was then frozen at −80° C. Subsequently, the pollen of each male was used to pollinate ears of each of four different inbreds (i.e., 4 females) that were unrelated to the males. Pollinations were conducted in the field in 2020 and, using the same male and female genotypes, similar pollinations were conducted in a greenhouse in February 2021.

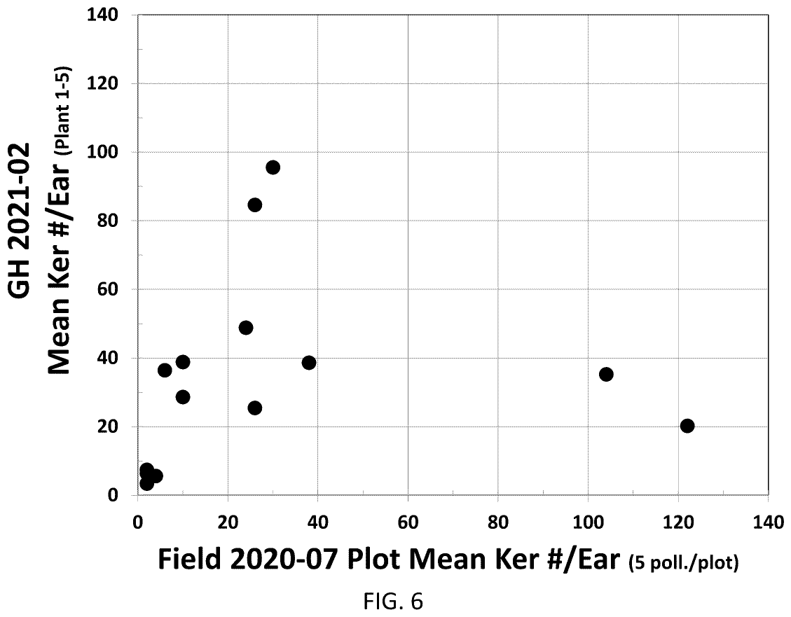

The results are shown in FIG. 6. Each point on FIG. 6 shows the average kernel number per ear for a specific male×female combination. The mean kernels per ear are plotted on the x axis for the field pollinations, while the mean kernels for the greenhouse pollinations are plotted on the y axis. Generally, seed set was less than 100 kernels per ear, which was significantly less than typical seed sets observed with cryogenically preserved pollen in 2019 experiments, however, the FBD fan speed used to dry the pollen used in the 2019 experiments was lower than the fan speed used in this experiment. The lower seed set in these experiments further demonstrates that the kinetic drying has a negative impact on pollen health. In particular, because the fan speed in the FBD was higher than the fan speed used for previous work, greater degrees of physical force impacted the pollen.

### Example 6: Dehydration Temperature

This experiment was designed to test the hypothesis that in addition to physical stress, dehydration temperature may also affect the retention of fertility in cryogenically preserved pollen. The experiment used maize pollen dried statically in RH boxes rather than dried in an FBD to ensure that the pollen was not subjected to motion-inflicted damage.

In this experiment, pollen was dried in an RH box at either 5° C., 12° C. or 20° C. for different lengths of time. Following drying, the pollen was placed into storage at −80° C. It is well known that when fresh pollen is stored for extended times, fertility of the pollen is best preserved when the pollen is stored under cool conditions (e.g., 5° C.), as compared to near-ambient temperatures (e.g., 20° C.). One might expect the same principles to apply when dehydrating pollen for sub-zero preservation, but the data in this experiment show that surprisingly, this is not the case.

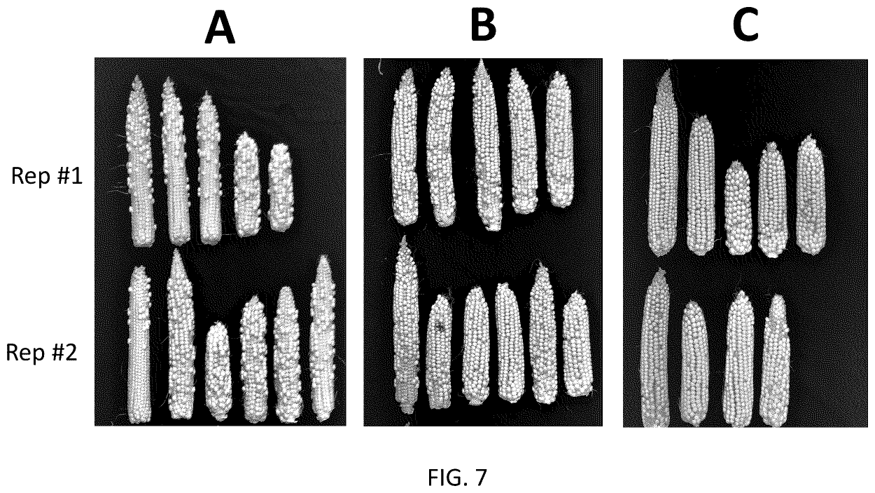

As seen in Table 2, seed set increased sharply for pollen dried at 12° C., versus pollen dried at 5° C., and seed set increased slightly more for pollen dried at 20° C. Representative ears from each treatment and each rep are shown in FIG. 7. However, samples in this experiment were dried for disparate lengths of time and had dissimilar PMCs following dehydration. Therefore, these data did not allow certainty that the observed differences in seed set were strictly related to the different temperatures of dehydration. As demonstrated in later experiments, the dehydration temperature is less critical than the RH and the VPD.

TABLE 2
Seed Set Comparison for Pollen
Dried at Different Temperatures

| | | | PMC | Mean + |
|---|---|---|---|---|
| | Incubation | Drying | After | SE |
| Treat- | Temperature | Duration | Drying | Kernels/ |
| ment | (° C.) | (hours) | (%) | Ear |
| A | 5 | 73 | 16.1 ± 0.4 | 106 ± 17 |
| B | 12 | 46 | 14.3 ± 1.0 | 312 ± 29 |
| C | 20 | 31 | 12.8 ± 0.0 | 354 ± 1 |

### Example 7: Dehydration Temperature

In order to confirm whether dehydration temperature was responsible for the differences in seed set, such as those seen in Example 6, similar additional experiments were conducted to assess the impact of dehydration temperature on maize pollen fertility.

Pollen was dried in RH boxes in preparation for cryogenic preservation by drying it at either 5° C. or 20° C. For each temperature, pollen was dried for different periods of time (from 11.1 to 14.4 hours) to achieve a fairly similar PMC (13.8% to 14.3%). The pollen was then frozen at −80° C. Subsequently, the pollen was used in pollinations.

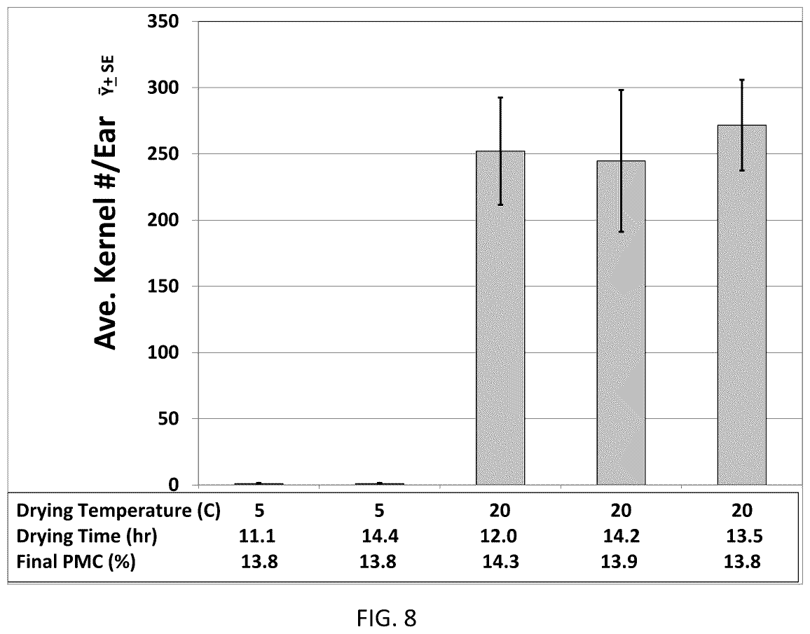

As shown in FIG. 8, seed set was abundant when the cryogenically preserved pollen was prepared for storage by drying at 20° C. In contrast, dehydrating the pollen at 5° C. prior to freezing resulted in pollen which exhibited very poor fertility and produced very few kernels per ear, despite the fact that the pollen used in both the 5° C. and 20° C. dehydration treatments was dried for a similar period of time and had comparable PMCs. While this experiment confirmed that drying temperature plays a role in viability, subsequent work showed that the more important factor is the RH and the VPD.

### Example 8: Optimum Moisture Level for Pollen Cryopreservation

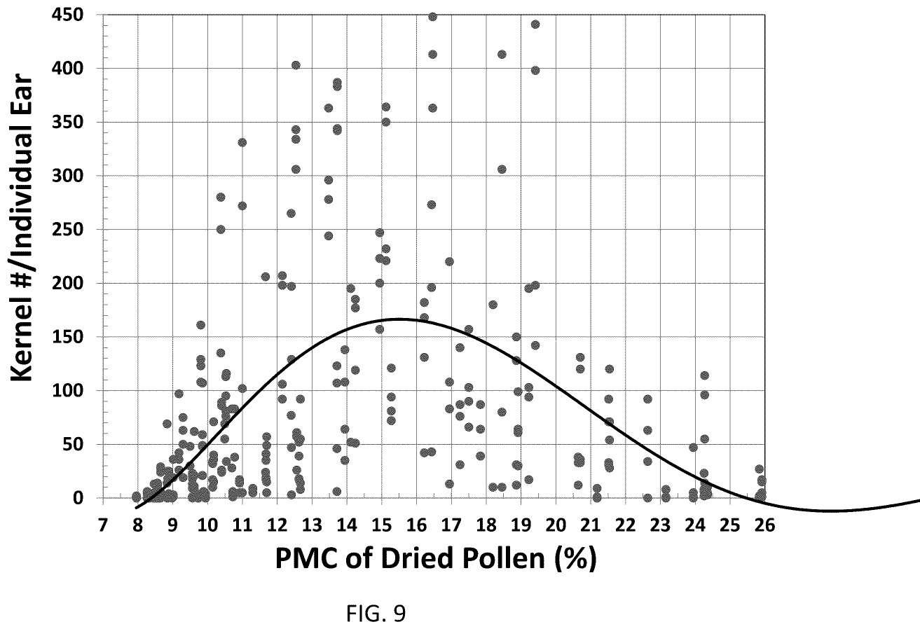

Experiments were conducted to determine the optimum pollen moisture content (PMC) target for maize pollen destined for cryopreservation. Data gathered from eight field experiments involving over 400 ears of corn pollinated by cryogenically preserved pollen. The pollen was gathered from four different males and was applied to ears of two different females. Pollen used in these experiments was prepared using kinetic drying in an FBD. It must be noted that in producing pollen with very low PMC values (e.g., <12%) in an FBD, the sample must be dried in the instrument for longer times than pollen possessing a moderately dry PMC (e.g., 15%). For the pollen of moisture concentrations less than 15%, poor seed set in these experiments is attributed to increased physical stress due to longer drying periods in an FBD as detailed in earlier examples. The data were plotted on a chart shown in FIG. 9, which demonstrates that the ideal PMC was determined to be very near to 15%.

### Example 9: Optimization of PMC in Static Drying

Following the determination that static drying of pollen resulted in pollen with better fertility than kinetically dried pollen, experiments were conducted to determine the optimal PMC for static drying.

Pollen was dried in static conditions at 20° C. in RH boxes to PMCs between 12.8% and 17.8%. The drying periods required to produce the pollen were generally similar. Following drying, the pollen was frozen at −80° C. The frozen pollen samples were then used to conduct pollinations of maize plants.

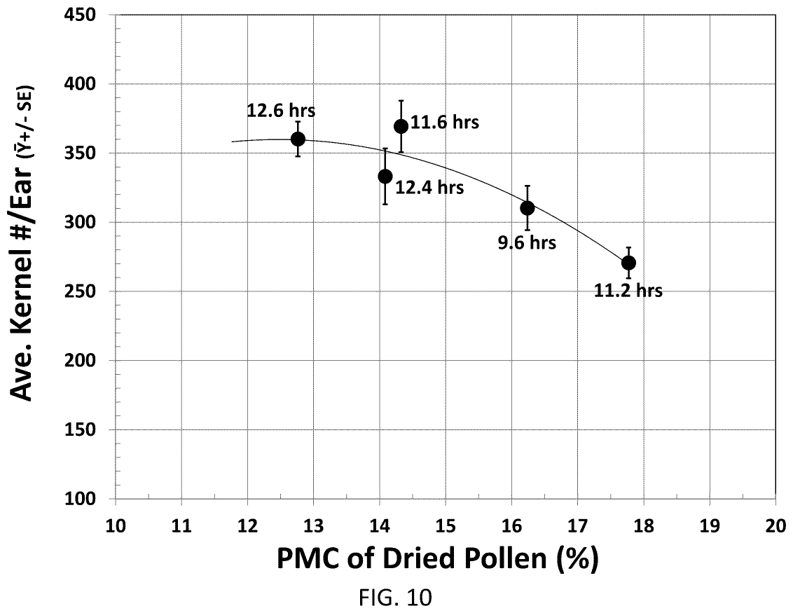

FIG. 10 shows the PMC of the dried pollen on the x axis and the average number of kernels generated by using the pollen on the y axis. The time to reach the PMC is provided in hours, next to each data point on the chart. The data demonstrate that the optimal PMC for cryogenically preserving statically dried pollen is roughly 12% to 15%. It is clear from the experiment that a PMC greater than approximately 15% disfavors the fertility of cryogenically preserved pollen. For example, pollen dried to 18% versus 15% yielded about 25% fewer kernels per ear.

### Example 10: Drying at Different VPD Levels

An experiment was designed to demonstrate the impact of different VPD levels on pollen fertility after cryogenic storage. Fresh pollen was dried in RH boxes at 20° C. under three disparate levels of initial VPD. The experiment was conducted such that all samples reached a PMC of 15.8%±0.6 within 10 hours, regardless of the starting VPD. Following drying, the samples were placed into storage at −80° C. Subsequently, the pollen samples were used to pollinate maize plants and the number of resulting kernels per ear were counted.

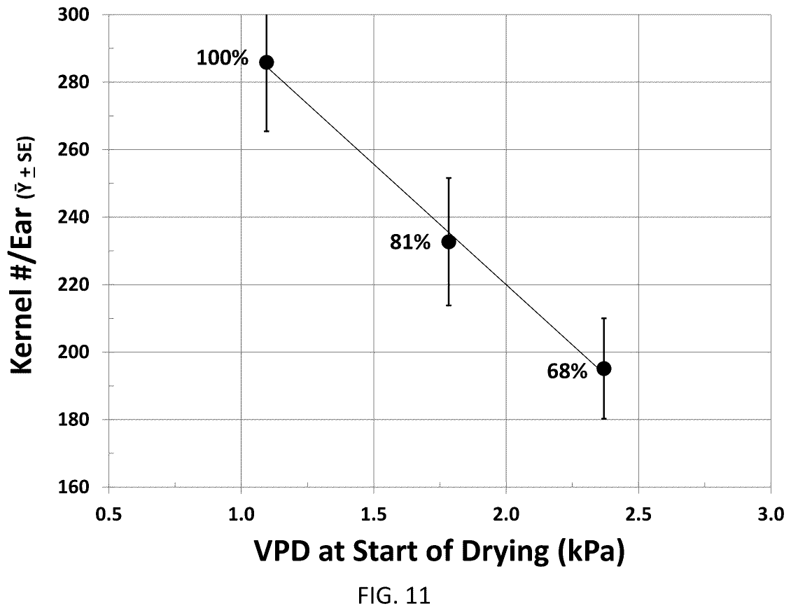

FIG. 11 plots VPD at the start of the drying period on the x axis, while the number of kernels resulting from pollinations using the pollen samples following preservation is on the y axis. The results show that a linear relationship exists between seed set on the ear and the VPD under which the pollen dried. The results further indicate that when dehydrating fresh pollen in preparation for cryogenic storage, the VPD under which the pollen is dried has a direct bearing on how productive the pollen will be when later used in pollinating ears.

### Example 11: Stability of Cryogenically Preserved Pollen Over Time

A pollen bank of six batches of dried and cryogenically preserved pollen was created in 2019, employing five male genotypes. The pollen bank was established in order to assess the stability of fertility in the dried and cryogenically preserved pollen over a period of five years. The PMC of the pollen samples is shown in Table 3.

TABLE 3
Pollen Bank Samples

| Pollen Bank | PMC of Pollen Bank |
|---|---|
| Sample | Sample at Time of |
| Designator | Cryopreservation |
| PL-65 | 17% |
| PL-66 | 18% |
| PL-67 | 19% |
| PL-68 | 16% |
| PL-69 | 15% |
| PL-70 | 13% |

Samples PL-67 and PL-69 were made using pollen of a single genotype, but from pollen collected and dried on different dates.

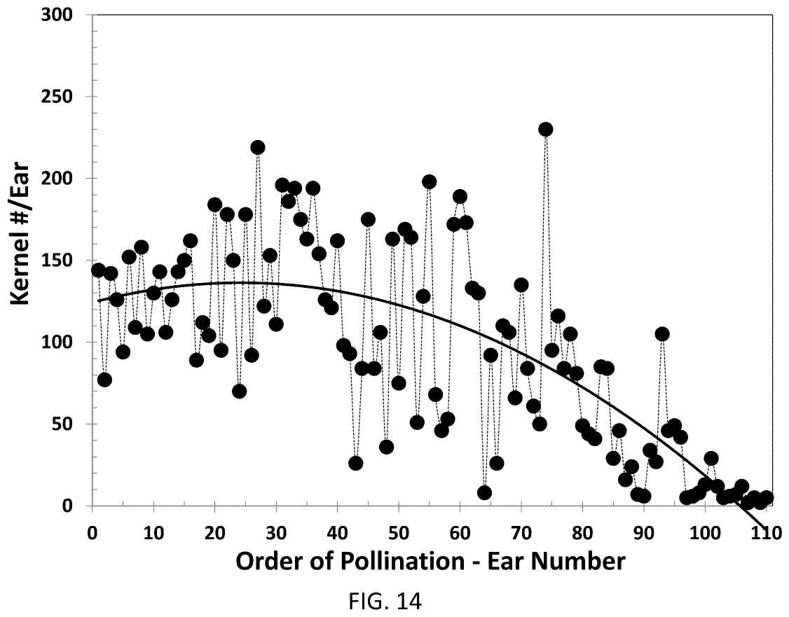

An overview of seed set on representative corn ears is shown in FIG. 14. The pollinations were made using pollen from the pollen bank made on five occasions across various females, which are listed on the left of FIG. 14. The results across the samples demonstrate that, in general, pollen samples that were stored with lower PMCs tended to produce greater seed set.

However, the genotype of the pollen may also be a factor in determining how well fertility is preserved in cryogenically preserved pollen. For instance, PL-67 and PL-69 samples were both made from inbred “C” but at the time of preservation, PL-69 had a PMC of 15% versus PL-67, which had a PLC of 19%. Despite having a higher PMC (19%), which generally is unfavorable to retention of fertility, PL-67 pollen still demonstrated moderate fertility. This may be attributable to inherent genetic factors which are unrelated to the influence dehydration PMC but are favorable in other ways to the long term cryogenic storage of pollen.

An additional effect of the pollen bank samples on seed set is the specific occasion on which the pollinations were made. For some samples, seed set was null on some pollination dates, whereas on other, later dates, seed set of sparse-to-moderate levels occurred. This signifies the importance of environmental factors in determining how effective cryogenically preserved pollen will be in successfully forming kernels.

### Example 12: Field Application of Dried and Cryogenically Preserved Pollen

An experiment to assess the use of dried and cryogenically preserved pollen in a field setting was conducted in 2021. A 50 mL polypropylene tube full of previously dried and cryogenically preserved pollen was loaded into a pollen gun that could produce a steady stream of pollen spray in a targeted, rate-controlled manner. This system was used to pollinate 110 ears in a timed fashion. Each ear was sprayed with two rapid bursts of pollen.

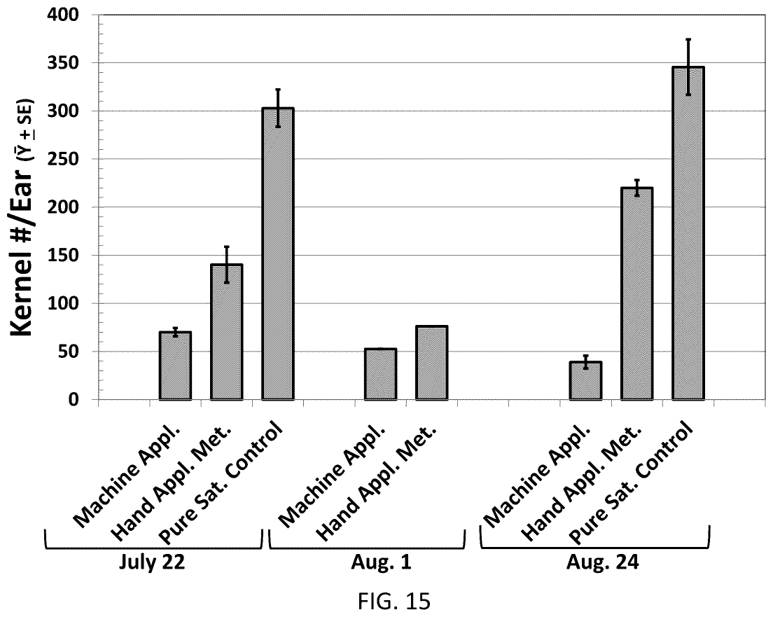

The duration of pollen gun applications was noted after the pollination of various ears (FIG. 15) and it was determined that approximately 10 ears were pollinated per minute.

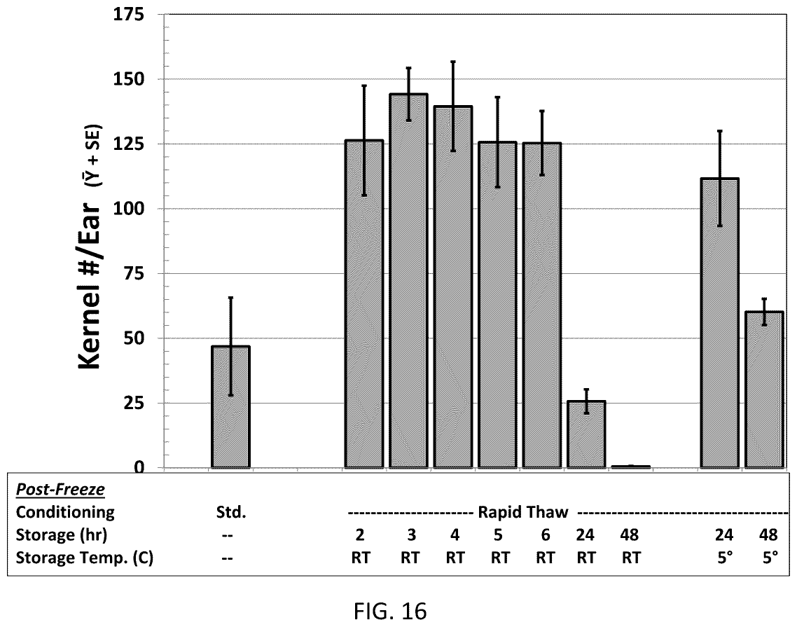

FIG. 16 shows the outcome of an examination of seed set on individual ears, with the ear number on the x axis and the number of kernels on each ear on the y axis. It can be seen that seed set began to decline after approximately 60 ears were pollinated (i.e., 6 minutes). Seed set dropped to near zero before pollen in the tube neared depletion, at 110 pollinations.

This experiment demonstrated that although the fertility of the dried and cryogenically preserved pollen is initially strong, the stability of the fertility began to deteriorate after approximately six minutes and declined to near zero within 10 minutes. Accordingly, dried and cryogenically preserved pollen does not typically have a long period of viability following thawing. This contributed to the need to develop stabilization processes for previously dehydrated and cryogenically frozen pollen.

### Example 13: Additional Field Application of Preserved Pollen

In 2022, additional experiments were conducted using dried and cryogenically preserved pollen in field conditions. Several treatments were used. In one treatment, pollen was applied to field rows of corn using an overhead system operated on a custom-retrofitted field sprayer. The pollen was sprayed towards fertile, silked ears from both sides of the plant. In another treatment, dried and cryogenically preserved pollen was applied by hand rather than using the pollen gun described in previous examples. A metered dose of dried and cryogenically preserved pollen was immediately administered (no stabilization techniques were used in this experiment) using a 1/64 tsp. scoop. Other ears were hand pollinated with a saturating dose of fresh pollen to serve as maximum potential controls (this treatment was not performed on the August 1 experiment). Negative controls were ears that were covered during the other applications.

The results of multiple such experiments are shown in FIG. 15. The values shown have been adjusted for the negative controls. The large-scale, machine application produced as much as a respectable average of 71 kernels per ear in a given experiment. The pollen administered by machine is administered in a ‘cloud’ and the targeting is not as accurate as hand pollinations, so the number of kernels of machine applied pollen is lower than that of the hand-applied, metered doses. The metered doses of dried and cryogenically preserved pollen produced a significant number of kernels from about 150 to about 225 kernels per ear. This is particularly impressive when compared to the positive controls which were a saturating dose, versus a small metered dose used for the test ears.

### Example 14: Post-Freeze Stabilization of Pollen

In the experiments described prior to this one, when using cryogenically frozen pollen for pollinations, the pollen was removed from ultra-cold storage and immediately applied to silks. In some cases, pollen is thawed prior to use but it is never stabilized to allow it to be used for a longer period after thawing. Multiple tests were conducted in the summer of 2022 to determine whether controlled thaw treatments and subsequent temperature controlled storage could improve the fertility of dried and cryopreserved pollen such that it could be stabilized and used for a longer time. The results of these experiments demonstrated that controlled thawing followed by incubation at cool temperatures improves the stability of previously dried and cryogenically preserved pollen. Even incubation at room temperature following controlled thawing can serve to stabilize pollen that has previously been dried and cryopreserved in accordance with the procedures outlined in this disclosure. Samples of dried and cryogenically preserved pollen used here were either: (a) immediately applied to silks after removal from dry ice (−78° C.) or, (b) rapidly thawed in a 40° C. water bath for two minutes, followed by incubation for various times at either room temperature (RT, approx. 22° C.) or in a refrigerator (approx. 5° C.).

The results shown in FIG. 16 demonstrate that the controlled thawing treatment followed by incubation to stabilize the pollen results in a greater degree of fertility from the previously dried and cryogenically preserved pollen. Seed set can be more than two-fold greater with pollen subjected to controlled thawing versus previous standard practice, such as that shown in Examples 12 and 13. The standard practice method result is shown in the first bar of FIG. 16. The remaining bars show the performance of pollen subjected to a rapid thaw followed by controlled storage at specific temperatures (room temperature or 5 degrees Celsius) for specific time frames. These results show that the increased fertility remains stable for longer periods of time than any demonstrated in the literature to date when the previously dehydrated and cryogenically frozen pollen is subjected to controlled thawing and then stored at either room temperature or refrigerated. For example, the stability of the pollen subjected to these stabilization techniques is extended by at least six hours when the samples are stored at room temperature. Furthermore, for previously dehydrated and cryogenically frozen pollen subjected to controlled thawing and storage at 5° C., fertility only declined 16% in 24 hours and still resulted in more than 50 kernels per ear from a metered pollination when used after 48 hours. This stabilization after thawing following dehydration and cryopreservation represents a very significant improvement over any previously known or disclosed treatment for retaining the viability of previously cryogenically preserved pollen.

It is anticipated that when bulk amounts of previously dehydrated and cryogenically frozen pollen are rapidly thawed, the level and stability of fertility will be increased to a degree similar to that observed with data in FIG. 16. This has profound implications for the prospects of commercially applying previously dehydrated and cryogenically frozen pollen. The stability of rapid-thawed pollen at room temperatures suggests that when commercial applications are made in the field and application periods at ambient conditions extend, for example, one to a few hours, there would be little concern that the fertility of the pollen is being compromised because of the holding temperature. In addition, our data indicate that if previously dehydrated and cryogenically frozen pollen is rapidly thawed in anticipation of large-scale application, but events (e.g., rainfall) occur that require postponement of pollen application, the thawed pollen can be held under refrigerated conditions and still applied to silks the next day with only a small change in the fertility of the pollen.

### Example 15: Drying Pollen Prior to Storage Improves Seed Set of Non-Cryogenically Stored Pollen

An experiment was conducted in Ankeny, IA in June 2023 to determine whether the benefits of drying pollen to levels used to prepare pollen for cryopreservation could also improve longevity of pollen stored at 5° C. and which was never subjected to any freezing temperatures.

Fresh maize pollen was collected at 59.6% moisture (w/w). A portion of the fresh pollen was dried at 33% RH and 30° C. following procedures similar to those described in Example 7 until the pollen reached a moisture content of 13.6% (w/w). Both the freshly collected pollen and dried pollen were mixed in a ratio of 4:1 diluent:pollen (w/w) with one of three PowerPollen® diluents designed to prevent the interaction of live pollen grains with dead pollen contents, in accordance with methods described in US Patent Application 20190008144. The mixtures were made in preparation for storage at 5° C. for up to 7 days. The pollen-diluent mixes containing fresh or dried pollen were sampled on days 3, 4, 5, 6, and 7 for viability based on kernel set. Pollen was applied in limiting amounts (a 1/64 tsp. scoop), which was a metered dose of pollen, to unpollinated silks of female maize inbreds W8039 after 3 and 4 days of storage, to unpollinated silks of female inbreds 2721 and R5357 after 5 days of storage, and to unpollinated silks of female inbred R5357 after 6 and 7 days of storage. In all cases, 7 or 8 female plants were pollinated 3 or 4 days after first silks appeared. Developing kernels per ear were determined at least 14 days after pollination.

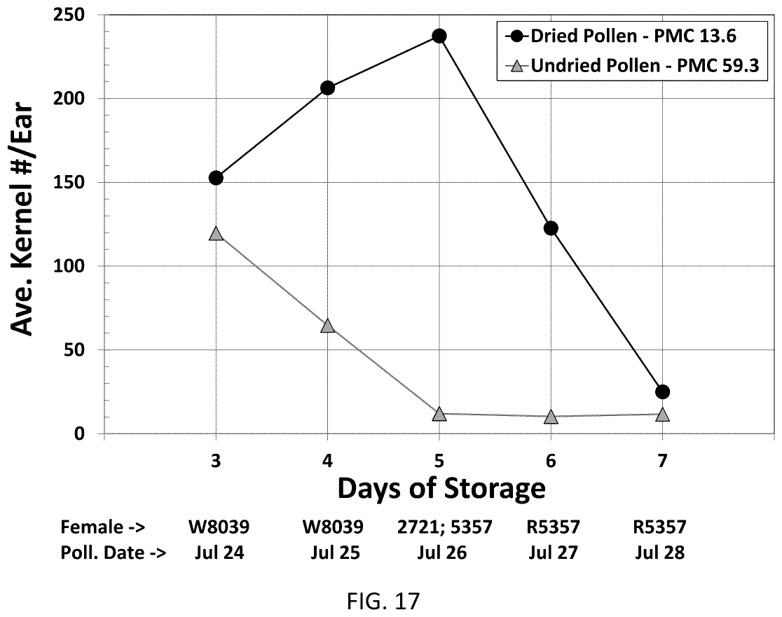

Data presented in FIG. 17 are pooled for the three diluent treatments. The results clearly show that drying pollen to 13.6% moisture prior to storage maintains the viability of pollen well beyond that of storing pollen at its original high moisture content. After 5 days of storage, pollinations with dried pollen produced nearly 250 kernels per ear, while pollinations with high moisture pollen failed to produce kernels above background levels. Declining levels of seed set observed after only 3 days of storage of high moisture pollen were extended to 6 days of storage for the dried pollen. These results indicate drying pollen to 13.6% moisture prior to storage extends viability dramatically even when the pollen is not stored in cryogenic conditions. The benefit of pollen drying was consistent across female genetics and diluent mixtures.

### Example 16: Interactive Effect of Temperature and Relative Humidity

We had demonstrated that the temperature maintained during dehydration of pollen as it was being prepared for cryopreservation affects the fertility retained in the dried and cryopreserved sample. Significantly greater seed set was obtained with pollen dried at 12 and 20° C., compared to pollen dried at 5° C. Furthermore, our experiments have demonstrated that when dehydrating fresh pollen in preparation for cryogenic storage, the evaporative demand under which the pollen is dried has a bearing on how productive the pollen will be when used in pollinating ears for seed production. Following an extensive search, no information was found in the public record of how pollen fertility may be systematically affected when pollen is dried at temperatures ranging above 20° C. Moreover, because evaporative demand during pollen dehydration was also proven to affect the fertility of dried, cryopreserved pollen, we sought to discover how both temperature (i.e., ≥20° C.) and RH of the drying environment interact to determine the relative degree of fertility preserved when pollen, which has been dehydrated and subsequently frozen at −80° C., is used to pollinate female reproductive organs.

A 3×3 factorial study was conducted whereby fresh maize pollen was dried at 20° C., 25° C., or 30° C. At each temperature, the pollen was dried in an RH box that contained a saturated salt solution of LiCl, MgCl₂, or NaBr. These salt solutions, in a sealed environment, produce an equilibrium RH of approximately 11%, 33%, or 55-59%, respectively. Because VPD is determined by both temperature and RH, this study dehydrated pollen at nine different VPDs (i.e., nine treatments).

For each treatment, pollen was dried until it reached a PMC of approximately 15%, at which time it was removed from the drying environment, mixed with a dry powder diluent (moisture content, 12-15%) in a deliberate proportion such that the ratio of pollen to diluent was known. This technique is in accordance with the technique described in Walden & Everett ((1961) Crop Sci 1: 21-25).

A small, metered quantity was aliquoted into a plastic tube ahead of placement in a −80° C. ultracold freezer. Upon removal from −80° C., the pollen was thawed at 40° C. for two minutes and then maintained at a cold but not freezing temperature in preparation for transfer to plants for pollination of ears. Nine or ten ears were pollinated for each treatment. Furthermore, the treatments were established, cryopreserved, and used for pollinations on three separate occasions, thereby establishing three replicates of the experiment. Pollinations were performed in a growth chamber May 16, 2023 and in the field on July 24 and Aug. 8, 2023.

Ahead of statistical analysis of the results, seed set data within each replicate of the experiment was normalized by dividing the kernel number of each ear by the average kernel number per ear across all ears of a given replicate. With this practice, the average normalized kernel number across all ears within a given replication of the experiment equals 1.00.

When averaging seed set within each temperature treatment, the normalized kernel number per ear was 0.92, 1.09, and 0.99 for 20° C., 25° C., and 30° C., respectively. Hence, drying pollen at temperatures between 20° C. and 30° C. does not have a large effect on the level of fertility present in the sample following cryopreservation, although drying at 25° C. produced the best result. In contrast, other studies of ours has shown that dehydration at 35° C. is detrimental to retention of fertility, relative to dehydration at 20° C. to 30° C. (data not shown).

In the 3×3 factorial study, and analogous to previous experiments, the fertility of dried, cryopreserved pollen always improved as the RH used for dehydration increased (mean normalized kernel number per ear: 0.43, 1.11, 1.46 for LiCl, MgCl₂, NaBr, respectively). The highly significant treatment effect of RH (p-value, <0.001) confirms the heretofore unreported importance of dehydrating pollen at an elevated RH (e.g., 55-60%) when aiming for the greatest preservation of fertility in dried, cryopreserved pollen samples. In our experience, pollen dried using a solution saturated with NaCl (equilibrium RH in sealed container, 75%) can also produce pollen of high fertility (data not shown).

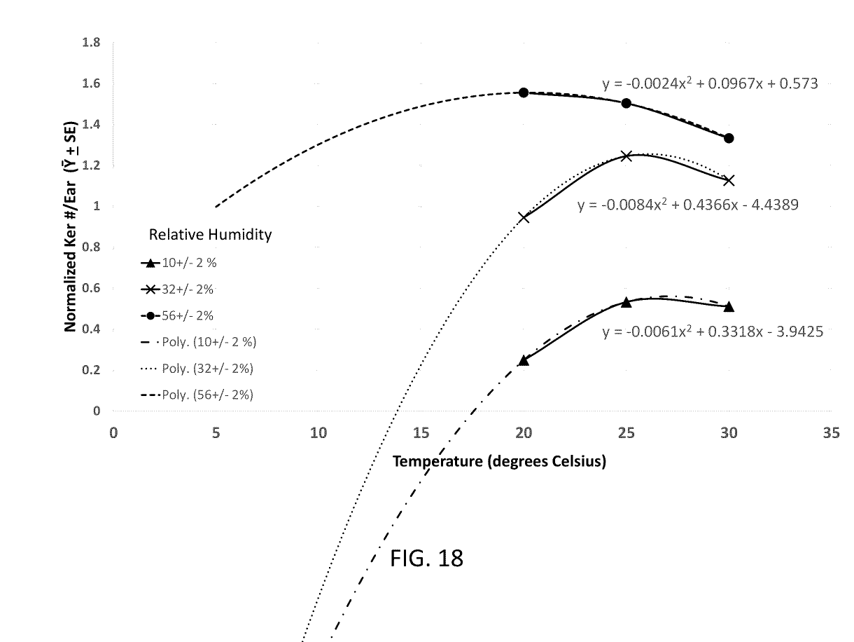

It important to note that, while the salt solution (i.e., RH) used for pollen dehydration had up to a 6-fold effect upon the fertility of dried, cryopreserved pollen, the precise importance of RH was dependent upon the temperature used for drying the pollen, as shown in FIG. 18. The interaction between temperature and RH was not statistically significant (p-value, 0.15), but nevertheless it plays an important role in dictating the temperature and RH one chooses for dehydrating pollen when maximal fertility following cryopreservation is desired. The occurrence of this interaction is also evident by the fact that when normalized seed set within each of the three replicates of the experiment was regressed in a quadratic fashion against the VPD of each of the nine treatments, the coefficients of correlation (R-square) were 0.14, 0.25, and 0.55. Therefore, the ability to predict retention of fertility based on the VPD used for pollen dehydration was poor due to the interaction between temperature and RH. In contrast, analogous regressions of VPD against drying time required to reach PMC 15% had R-square values of 0.84, 0.95, and 0.95, demonstrating the reproducibility of the pollen dehydration process.

The 3×3 factorial study demonstrated that drying pollen (for cryopreservation) at 20° C. in an environment of roughly 60% RH provided maximum seed set. By examining a broader range of temperatures and RHs, one of skill in the art can easily identify a particular temperature/RH combination that produces optimal seed set from cryopreserved pollen (e.g., dehydration at 15° C. and RH 70%). Anticipating this interaction between drying temperature and RH allows one to establish dehydration conditions that should be employed to preserve the greatest level of fertility in cryopreserved pollen.

FIG. 18 shows the outcome of the 3×3 factorial study. The y axis shows the normalized number of kernels per ear, while the x axis shows the temperature in degrees Celsius. Each RH treatment generates a separate seed set curve, indicative of pollen viability. The calculated equation for each curve has been plotted for each of the treatments and the equation for each curve is extended to show the anticipated results of drying at lower temperatures.

1. A method of preserving Poaceae pollen, comprising:
a. drying said pollen in controlled conditions wherein the movement of the pollen is minimized; and
b. storing said pollen, wherein at least a portion of said pollen remains viable during and after said storage and can be used to successfully pollinate and fertilize Poaceae plants.

2. The method of claim 1 wherein said storing is conducted at temperatures below 0 degrees Celsius.

3. The method of claim 1 wherein said storing is conducted at temperatures below −20 degrees Celsius.

4. The method of claim 1 wherein said storing is conducted at about −80 degrees Celsius.

5. The method of claim 1 wherein said storing is conducted at temperatures between 0 degrees Celsius and 10 degrees Celsius.

6. The method of claim 1 wherein said storing is conducted at temperatures above 10 degrees Celsius.

7. The method of claim 1 wherein during the drying step, the pollen is gently moved to prevent aggregation of pollen grains.

8. The method of claim 1 wherein said pollen is dried to a pollen moisture content ranging from 10% to 22%.

9. The method of claim 8 wherein said pollen is dried to a pollen moisture content of about 15%.

10. The method of claim 1 wherein the rate at which said pollen is dried is influenced by one or more of the following parameters:
a. relative humidity
b. vapor pressure deficit
c. airflow
d. amount of pollen being dried
e. starting pollen moisture content
f. radiant energy
g. temperature

11. The method of claim 10 wherein one or more of said parameters is adjustable.

12. The method of claim 10 wherein the temperature ranges from 5 degrees Celsius to 30 degrees Celsius.

13. The method of claim 10 wherein the temperature ranges from 15 degrees Celsius to 25 degrees Celsius.

14. The method of claim 10 wherein the temperature ranges from 24 degrees Celsius to 25 degrees Celsius.

15. The method of claim 10 wherein the vapor pressure deficit ranges from 0.1 kPa to 2.0 kPa.

16. The method of claim 1 further comprising applying said stored pollen to the stigmas of a species of Poaceae plant capable of pollination and fertilization by the stored species of Poaceae pollen.

17. The method of claim 1 further comprising stabilizing the pollen by thawing it, such that the full volume of frozen pollen is thawed within a period ranging from 10 seconds to 300 seconds.

18. The method of claim 17 wherein the step of thawing the frozen pollen includes exposing the frozen pollen to a temperature ranging from 36 degrees Celsius to 44 degrees Celsius.

19. The method of claim 17 wherein the frozen pollen is exposed to a water bath wherein the water is at a temperature ranging from 37 degrees Celsius to 42 degrees Celsius.

20. The method of claim 17 further comprising applying said thawed pollen to the stigmas of a species of Poaceae plant capable of pollination and fertilization by the species of thawed Poaceae pollen.

21. The method of claim 20 wherein said thawed pollen is further stabilized by storing it at a temperature above 0 degrees Celsius for up to twenty-four hours before application to said Poaceae plant.

22. The method of claim 21 wherein the storage occurs at a temperature ranging from 1 degree Celsius to 23 degrees Celsius.

23. The method of claim 20 wherein the application of thawed pollen results in Poaceae seeds.

24. The method of claim 23 wherein said Poaceae seeds are intended for seed production purposes.

25. The method of claim 20 wherein the amount of thawed pollen applied to said Poaceae plant results in about 31-100% of the number of seeds that would have resulted from application of the same amount of fresh pollen.

26. The method of claim 24 wherein the amount of thawed pollen applied to said Poaceae plant results in about 50-100% of the number of seeds that would have resulted from application of the same amount of fresh pollen.

27. The method of claim 25 wherein the amount of thawed pollen applied to said Poaceae plant results in about 50-90% of the number of seeds that would have resulted from application of the same amount of fresh pollen.

28. The method of claim 1 wherein said drying step occurs in a relative humidity box.

29. The method of claim 1 wherein said minimization of pollen movement reduces damage to the Poaceae pollen in comparison to pollen preservation methods that do not minimize pollen movement.

30. The method of claim 1 wherein the pollen moisture removal is controlled to result in a predetermined rate of drying.

31. A method of stabilizing cryogenically stored Poaceae pollen comprising
a. drying said pollen in controlled conditions wherein the movement of the pollen is minimized;
b. subjecting said pollen to cryogenic storage;
c. rapidly thawing the Poaceae pollen such that the temperature of the pollen exceeds 0 degrees Celsius; and
d. stabilizing the thawed Poaceae pollen by maintaining it at a temperature above 0 degrees Celsius and below 40 degrees Celsius;
wherein the pollen grains maintain viability such that they can be used to successfully pollinate and fertilize the species of Poaceae plant from which the pollen was collected.

32. The method of claim 31 wherein the thawing of the Poaceae pollen is conducted in a water bath.

33. The method of claim 32 wherein the water bath is at a temperature ranging from 36 degrees Celsius to 44 degrees Celsius.

34. A composition comprising Poaceae pollen that has been dried in conditions wherein the movement of said pollen is minimized, which is subsequently stored at a temperature such that at least a portion of said pollen remains viable during and after said storage and can be used to successfully pollinate and fertilize Poaceae plants.

35. The composition of claim 34 wherein said storage is conducted at temperatures below 0 degrees Celsius.

36. The composition of claim 34 wherein said storage is conducted at temperatures below −20 degrees Celsius.

37. The composition of claim 34 wherein said storage is conducted at about −80 degrees Celsius.

38. The composition of claim 34 wherein said storage is conducted at temperatures between 0 degrees Celsius and 10 degrees Celsius.

39. The composition of claim 34 wherein said storage is conducted at temperatures above 10 degrees Celsius.

40. The composition of claim 34 wherein said pollen is dried to a pollen moisture content ranging from 10% to 22%.

41. The composition of claim 40 wherein said pollen is dried to a pollen moisture content of about 15%.

42. A method of applying the composition of claim 34 to the stigmas of a species of Poaceae plant capable of pollination by the species of Poaceae pollen contained in the composition.

43. The method of claim 42 wherein the composition of claim 34 is stored for up to twenty-four hours at a temperature above 0 degrees Celsius before application to said Poaceae plant.

44. The method of claim 42 wherein the application of the composition of claim 34 results in Poaceae seeds.

45. A method of stabilizing the composition of claim 34 comprising
a. drying said pollen in controlled conditions wherein the movement of the pollen is minimized;
b. subjecting said pollen to cryogenic storage;
c. rapidly thawing the Poaceae pollen such that the temperature of the pollen exceeds 0 degrees Celsius; and
d. stabilizing the thawed Poaceae pollen by maintaining it at a temperature above 0 degrees Celsius and below 40 degrees Celsius;
wherein the pollen grains maintain viability such that they can be used to successfully pollinate and fertilize the species of Poaceae plant from which the pollen was collected.
